(19) United States
(12) Patent Application Publication
(10) Pub. No.: US 2026/0257980 A1
(43) Pub. Date: Sep. 3, 2026

(54) NOVEL DIMETHYLCHALCONE DERIVATIVE

(71) Applicants: CHUNG-ANG UNIVERSITY INDUSTRY-ACADEMIC COOPERATION FOUNDATION, Seoul (KR); SEOUL NATIONAL UNIVERSITY R & DB FOUNDATION, Seoul (KR); SEOUL NATIONAL UNIVERSITY HOSPITAL, Seoul (KR)

(72) Inventors: Kwang Yong PARK, Seoul (KR); Kyong Soo PARK, Seoul (KR)

(73) Assignees: CHUNG-ANG UNIVERSITY INDUSTRY-ACADEMIC COOPERATION FOUNDATION, Seoul (KR); SEOUL NATIONAL UNIVERSITY R & DB FOUNDATION, Seoul (KR); SEOUL NATIONAL UNIVERSITY HOSPITAL, Seoul (KR)

(21) Appl. No.: 18/846,718

(22) PCT Filed: Mar. 14, 2023
(86) PCT No.: PCT/KR2023/003430

§ 371 Date: Sep. 13, 2024

(30) Foreign Application Priority Data
Mar. 15, 2022 (KR) 10-2022-0032393
Mar. 14, 2023 (KR) 10-2023-0033415

(51) Int. Cl. C07C 43/23; A61K 31/12; A61P 3/00; C07C 43/225

(52) U.S. Cl. CPC C07C 43/23; A61K 31/12; A61P 3/00; C07C 43/225

(57) ABSTRACT

Provided are novel compounds based on the meta-substitution of 2′,4′-dihydroxy-6′-methoxy-3′,5′-dimethylchalcone (DMC). According to the presently claimed subject matter, novel DMC derivatives that are meta-substituted using cheap phloroglucinol or the like as a starting material can be simply and efficiently synthesized. Specifically, DMC derivatives according to the presently claimed subject matter were confirmed to have a good fatty acid oxidation effect compared to existing DMC derivatives, and thus are expected to be used as a therapeutic material for metabolic diseases.

### TECHNICAL FIELD

The present invention relates to novel dimethylchalcone (DMC) derivatives, and the like.

This application claims priority to and the benefit of Korean Patent Application Nos. 10-2022-0032393 and 10-2023-0033415 filed on Mar. 15, 2022 and Mar. 14, 2023, respectively, and all the contents disclosed in the specification and drawings of the applications are incorporated in this application.

### BACKGROUND ART

Various materials extracted from natural plants have had a great impact on the treatment of human diseases. However, in the case of intake of natural products, their quantity is limited compared to demand, and it is not possible to change side effects or efficacy by adjusting specific ingredients. Therefore, it is necessary to produce medicines that can replace natural products through total synthetic research. 2′,4′-dihydroxy-6′-methoxy-3′,5′-dimethylchalcone (DMC) is a chalcone-based compound that has an aromatic ketone backbone. DMC is typically extracted from the flower buds of Cleistocalyx operculatus, and has been reported to have antitumor activity, and the like.

DMC having high biological value is receiving much attention and various pharmacological studies are being conducted on DMC are being conducted. However, to date, research has only been conducted on DMC itself, and derivatives of DMC compounds and their properties are not yet clearly known.

Also, since Cleistocalyx operculatus itself is sparsely distributed, and the extraction content of DMC in Cleistocalyx operculatus is very low, there are limits to conducting various pharmacological studies on DMC due to its insufficient production amount. Therefore, there is a need for research on a new manufacturing method that can design an economical and efficient synthetic route for DMC and DMC derivatives using inexpensive reactants, produce various derivatives through changes in functional groups, and maximize the efficacy of DMC and derivatives thereof.

Meanwhile, a metabolic disease refers to a disease in which various symptoms such as obesity, diabetes, hypertension, hyperlipidemia, coronary or arteriosclerosis, non-alcoholic fatty liver, and the like develop simultaneously due to chronic metabolic disorders, and it was first identified by Reaven in 1988. Metabolic diseases are characterized by insulin resistance, hypertension, dyslipidemia, and the like, and most of them are accompanied by increased body weight or obesity. The most serious problem of metabolic diseases is the onset of chronic complications such as diabetic retinopathy, diabetic nephropathy, diabetic foot disease, diabetic neuropathy, hyperlipidemia, cardiovascular disease (stroke, angina pectoris, myocardial infarction, peripheral vascular disease), and the like. Most of these chronic complications proceed irreversibly once they occur, and since there is still no way to completely block this process, these chronic complications cause serious symptoms and even lead to the death of a patient when not appropriately treated. Also, to date, patients have taken hypoglycemic agents, antihypertensive agents, cholesterol-lowering agents, and the like individually in order to treat metabolic diseases having these complex symptoms. Therefore, there is a need for developing a novel therapeutic agent capable of simultaneously treating various symptoms in order to effectively manage and treat metabolic diseases having these complex symptoms.

### DISCLOSURE

### Technical Problem

The present invention is designed to solve the above problems of the related art, and has been completed by confirming that novel meta-substituted DMC derivatives may be simply and efficiently synthesized using inexpensive phloroglucinol or the like as a starting material, and may be used as a therapeutic material for metabolic diseases and the like because the DMC derivatives have excellent fatty acid oxidation effects. Therefore, the present invention has been completed based on the above facts.

Accordingly, it is an object of the present invention to provide a novel DMC derivative.

It is another object of the present invention to provide a pharmaceutical composition for preventing or treating a metabolic disease, which includes the DMC derivative as an active ingredient.

However, the technical objects to be achieved by the present invention are not limited to the above-described technical objects, and other objects which are not mentioned above will be clearly understood from the following detailed description by those skilled in the art to which the present invention pertains.

### Technical Solution

According to an aspect of the present invention, there is provided a compound represented by the following Chemical Formula I, an isomer thereof, or a pharmaceutically acceptable salt thereof.

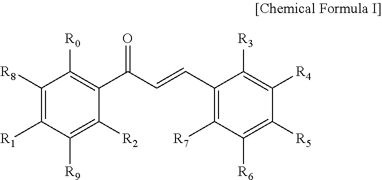

wherein:
R₀, R₁, and R₂ are each independently a hydroxyl group (OH), a methoxymethoxy group (OCH₂OCH₃; OMOM), or a C₁-C₁₀ alkoxy group;
R₈ and R₉ are each independently a C₁-C₁₀ alkyl group;
R₃ to R₇ are each independently any one selected from the group consisting of a halogen, hydrogen (H), deuterium (D), a thiol group (SH), a cyano group (CN), a nitro group (NO₂), a substituted or unsubstituted amino group (NH₂), a substituted or unsubstituted C₁-C₁₀ alkylthio group, a substituted or unsubstituted C₁-C₁₀ alkylsulfonyl group, a substituted or unsubstituted C₁-C₁₀ alkylsulfoxy group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₆-C₂₀ aryl group; and
the term “substituted or unsubstituted” refers to being unsubstituted or substituted with one or more substituents selected from the group consisting of a halogen group, a nitrile group, a nitro group, a hydroxyl group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, an alkylsulfonyl group, an arylsulfonyl group, a silyl group, a boron group, an alkyl group, a cycloalkyl group, an alkenyl group, an alkynyl group, an aryl group, an aralkyl group, an aralkenyl group, an alkylaryl group, an alkylamine group, an aralkylamine group, a heteroarylamine group, an arylamine group, an arylphosphine group, and a heterocyclic group;
provided that at least any one of R₄ and R₆ is not hydrogen;
at least any one of R₀, R₁, and R₂ is not OH; and
when R₀ and R₁ are both OMe or both OMOM, R₂ is not OH or OMOM.

According to another exemplary embodiment of the present invention, R₈ and R₉ may each be a methyl group, but the present invention is not limited thereto.

According to still another exemplary embodiment of the present invention, R₈ and R₉ may each be a methyl group, and R₀ may be OMe, but the present invention is not limited thereto.

According to yet another exemplary embodiment of the present invention, the compound may be represented by the following Chemical Formula I-1, but the present invention is not limited thereto:

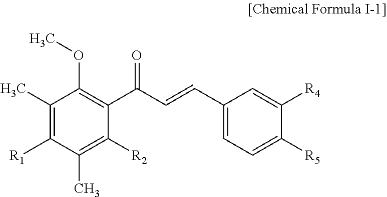

wherein:
R₄ is not hydrogen; and
R₁ and R₂ are each independently OH, OMOM, or a C₁-C₁₀ alkoxy group;
provided that when R₁ is OMe, R₂ is not OH or OMOM.

According to yet another exemplary embodiment of the present invention, the compound may be represented by the following Chemical Formula I-2, but the present invention is not limited thereto:

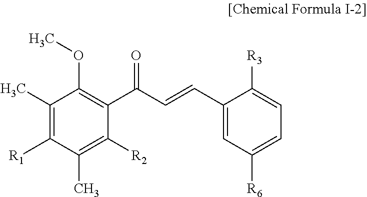

wherein:
R₆ is not hydrogen; and
R₁ and R₂ are each independently OH, OMOM, or a C₁-C₁₀ alkoxy group;
provided that when R₁ is OMe, R₂ is not OH or OMOM.

According to yet another exemplary embodiment of the present invention, the compound may be represented by the following Chemical Formula I-3, but the present invention is not limited thereto:

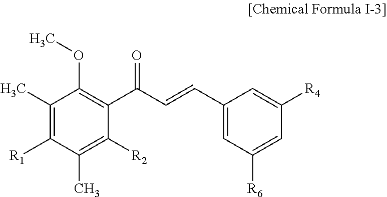

wherein:
R₆ is not hydrogen;
R₁ and R₂ are each independently OH, OMOM, or a C₁-C₁₀ alkoxy group;
provided that when R₁ is OMe, R₂ is not OH or OMOM.

The compound may be selected from the group consisting of the following, but the present invention is not limited thereto:
(1) 1-(2′-hydroxy-4′-(methoxymethoxy)-6′-methoxy-3′,5′-dimethylphenyl)-3-(3-fluorophenyl)-2-propen-1-one;
(2) 1-(2′-hydroxy-4′-(methoxymethoxy)-6′-methoxy-3′,5′-dimethylphenyl)-3-(3,4-difluorophenyl)-2-propen-1-one;
(3) 1-(2′-hydroxy-4′-(methoxymethoxy)-6′-methoxy-3′,5′-dimethylphenyl)-3-(3-trifluoromethylphenyl)-2-propen-1-one;
(4) 1-(2′-hydroxy-4′-(methoxymethoxy)-6′-methoxy-3′,5′-dimethylphenyl)-3-(2,5-difluorophenyl)-2-propen-1-one;
(5) 1-(2′-hydroxy-4′-(methoxymethoxy)-6′-methoxy-3′,5′-dimethylphenyl)-3-(3,5-difluorophenyl)-2-propen-1-one;
(6) 1-(2′-hydroxy-4′-(methoxymethoxy)-6′-methoxy-3′,5′-dimethylphenyl)-3-(3,5-dimethylphenyl)-2-propen-1-one;
(7) 1-(2′-hydroxy-4′-(methoxymethoxy)-6′-methoxy-3′,5′-dimethylphenyl)-3-(3-bromophenyl)-2-propen-1-one;
(8) 1-(2′,4′-dihydroxy-6′-methoxy-3′,5′-dimethylphenyl)-3-(3-fluorophenyl)-2-propen-1-one;
(9) 1-(2′,4′-dihydroxy-6′-methoxy-3′,5′-dimethylphenyl)-3-(3,4-difluorophenyl)-2-propen-1-one;
(10) 1-(2′,4′-dihydroxy-6′-methoxy-3′,5′-dimethylphenyl)-3-(3-trifluoromethylphenyl)-2-propen-1-one;
(11) 1-(2′,4′-dihydroxy-6′-methoxy-3′,5′-dimethylphenyl)-3-(2,5-difluorophenyl)-2-propen-1-one;
(12) 1-(2′,4′-dihydroxy-6′-methoxy-3′,5′-dimethylphenyl)-3-(3,5-difluorophenyl)-2-propen-1-one;
(13) 1-(2′,4′-dihydroxy-6′-methoxy-3′,5′-dimethylphenyl)-3-(3,5-dimethylphenyl)-2-propen-1-one;
(14) 1-(2′,4′-dihydroxy-6′-methoxy-3′,5′-dimethylphenyl)-3-(3-bromophenyl)-2-propen-1-one;
(15) 1-(2′,4′,6′-trimethoxy-3′,5′-dimethylphenyl)-3-(3-fluorophenyl)-2-propen-1-one;
(16) 1-(2′,4′,6′-trimethoxy-3′,5′-dimethylphenyl)-3-(3,4-difluorophenyl)-2-propen-1-one;
(17) 1-(2′,4′,6′-trimethoxy-3′,5′-dimethylphenyl)-3-(3-(trifluoromethyl)phenyl)-2-propen-1-one; and
(18) 1-(2′,4′,6′-trimethoxy-3′,5′-dimethylphenyl)-3-(2-chloro-5-(trifluoromethyl)phenyl)-2-propen-1-one.

According to another aspect of the present invention, there is provided a pharmaceutical composition for preventing or treating a metabolic disease, which includes the compound according to the present invention, the isomer thereof, or the pharmaceutically acceptable salt thereof as an active ingredient.

According to still another aspect of the present invention, there is provided a pharmaceutical kit for preventing or treating a metabolic disease, which includes the compound according to the present invention, an isomer thereof, or a pharmaceutically acceptable salt thereof as an active ingredient.

According to yet another aspect of the present invention, there is provided a food composition for preventing or ameliorating a metabolic disease, which includes the compound according to the present invention, an isomer thereof, or a sitologically acceptable salt thereof as an active ingredient. The food composition includes a health functional food composition.

According to yet another aspect of the present invention, there is provided a use of the compound according to the present invention, an isomer thereof, or a pharmaceutically acceptable salt thereof for the prevention or treatment of a metabolic disease.

According to yet another aspect of the present invention, there is provided a method of preventing or treating a metabolic disease, which includes administering the compound according to the present invention, an isomer thereof, or a pharmaceutically acceptable salt thereof to a subject in need thereof. The compound according to the present invention, an isomer thereof, or a pharmaceutically acceptable salt thereof may be administered in an effective amount.

According to yet another aspect of the present invention, there is provided a use of the compound according to the present invention, an isomer thereof, or a pharmaceutically acceptable salt thereof for the manufacture of a medicine for treating a metabolic disease

According to one exemplary embodiment of the present invention, the metabolic disease may be any one or more selected from the group consisting of obesity, diabetes, hypertension, hyperlipidemia, arteriosclerosis, acute coronary syndrome, non-alcoholic fatty liver, diabetic retinopathy, diabetic nephropathy, diabetic foot disease, diabetic neuropathy, hyperlipidemia, stroke, angina pectoris, myocardial infarction, and peripheral vascular disease, but the present invention is not limited thereto.

### Advantageous Effects

The present invention relates to novel compounds based on the meta-substitution of 2′,4′-dihydroxy-6′-methoxy-3′,5′-dimethylchalcone (DMC). According to the present invention, novel meta-substituted DMC derivatives can be simply and efficiently synthesized using inexpensive phloroglucinol or the like as a starting material. In particular, the DMC derivatives according to the present invention are expected to be used as a therapeutic material for metabolic diseases because the DMC derivatives were confirmed to have superior fatty acid oxidation effects compared to existing DMC derivatives.

### DESCRIPTION OF DRAWINGS

FIG. 1 shows the results of confirming the effect of DMC derivatives according to one embodiment of the present invention on fatty acid oxidation.

FIGS. 2 to 4 show structural formulas of the DMC derivatives according to one embodiment of the present invention.

FIG. 5 shows the structural formulas of the control compounds used in Example 4.

### BEST MODE

The present invention is directed to providing a novel compound based on the meta-substitution of 2′,4′-dihydroxy-6′-methoxy-3′,5′-dimethylchalcone (DMC), an isomer thereof, and a pharmaceutically acceptable salt thereof.

Throughout the specification, the term “compound represented by Chemical Formula N” may be simply expressed as “compound N,” “chemical formula N,” or (N).

The compound according to the present invention may be represented by the following Chemical Formula I, but the present invention is not limited thereto.

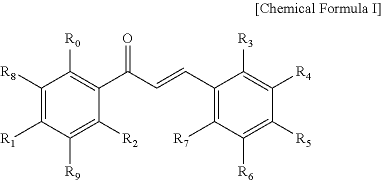

wherein:
R₀, R₁, and R₂ are each independently a hydroxyl group (OH), a methoxymethoxy group (OCH₂OCH₃; OMOM), or a C₁-C₁₀ alkoxy group;
R₈ and R₉ are each independently a C₁-C₁₀ alkyl group;
R₃ to R₇ are each independently any one selected from the group consisting of a halogen, hydrogen (H), deuterium (D), a thiol group (SH), a cyano group (CN), a nitro group (NO₂), a substituted or unsubstituted amino group (NH₂), a substituted or unsubstituted C₁-C₁₀ alkylthio group, a substituted or unsubstituted C₁-C₁₀ alkylsulfonyl group, a substituted or unsubstituted C₁-C₁₀ alkylsulfoxy group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₆-C₂₀ aryl group; and
the term “substituted or unsubstituted” refers to being unsubstituted or substituted with one or more substituents selected from the group consisting of a halogen group, a nitrile group, a nitro group, a hydroxyl group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, an alkylsulfonyl group, an arylsulfonyl group, a silyl group, a boron group, an alkyl group, a cycloalkyl group, an alkenyl group, an alkynyl group, an aryl group, an aralkyl group, an aralkenyl group, an alkylaryl group, an alkylamine group, an aralkylamine group, a heteroarylamine group, an arylamine group, an arylphosphine group, and a heterocyclic group;
provided that at least any one of R₄ and R₆ is not hydrogen;
at least any one of R₀, R₁, and R₂ is not OH; and
when R₀ and R₁ are both OMe or both OMOM, R₂ is not OH or OMOM.

According to one exemplary embodiment of the present invention, R₀, R₁, and R₂ may each independently be a hydroxyl group (OH), a methoxy group (OCH₃; OMe), a methoxymethoxy group (OCH₂OCH₃; OMOM), or a C₁-C₁₀ alkoxy group, but the present invention is not limited thereto.

According to one exemplary embodiment of the present invention, at least one of R₀, R₁ and R₂ may be OMe, and at least two or more of R₀, R₁ and R₂ may be simultaneously OH, OMOM, or a C₁-C₁₀ alkoxy group, but the present invention is not limited thereto.

According to another exemplary embodiment of the present invention, R₈ and R₉ may each independently be a C₁-C₁₀ alkyl group or a methyl group, but the present invention is not limited thereto. Preferably, R₈ and R₉ may be the same. Most preferably, R₈ and R₉ are each a methyl group.

According to still another exemplary embodiment of the present invention, R₀ may be a C₁-C₁₀ alkoxy group or OMe, but the present invention is not limited thereto.

According to yet another exemplary embodiment of the present invention, the compound may be represented by the following Chemical Formula I-1, Chemical Formula I-2, or Chemical Formula I-3, but the present invention is not limited thereto:

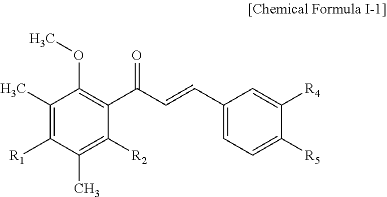

wherein:
R₄ is not hydrogen; and
R₁ and R₂ are each independently OH, OMOM, or a C₁-C₁₀ alkoxy group;
provided that when R₁ is OMe, R₂ is not OH or OMOM;

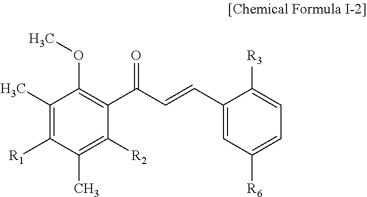

wherein:
R₆ is not hydrogen; and
R₁ and R₂ are each independently OH, OMOM, or a C₁-C₁₀ alkoxy group;
provided that when R₁ is OMe, R₂ is not OH or OMOM; and

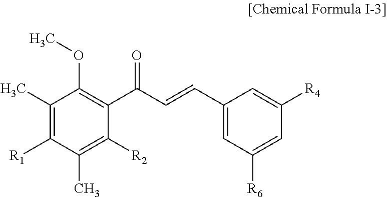

wherein:
R₆ is not hydrogen; and
R₁ and R₂ are each independently OH, OMOM, or a C₁-C₁₀ alkoxy group;
provided that when R₁ is OMe, R₂ is not OH or OMOM.

Preferably, the compound according to the present invention is characterized as being a derivative of 2′,4′-dihydroxy-6′-methoxy-3′,5′-dimethylchalcone (DMC). In the present invention, “DMC derivatives” may include any derivative derived from DMC without limitation. Preferably, the DMC derivatives may be dimethylchalcone derivatives having variously substituted chalcone structures using phloroglucinol as a starting material. More preferably, the compound according to the present invention may be a DMC derivative having one or more meta-substituents. Preferably, the meta-substituents are present in the right-hand ring of Chemical Formula I.

Most preferably, the compound according to the present invention may be any one of 18 compounds as shown in Table 1 below.

In the present invention, a halogen may include F, Cl, Br, I, or the like.

In the present invention, the alkyl refers to a completely saturated, branched or non-branched (or, straight or linear) hydrocarbon. In the present invention, the alkyl may be a C₁-C₂₀, C₁-C₁₅, C₁-C₁₂, C₁-C₁₀, C₁-C₈, C₁-C₆, C₁-C₅, or C₁-C₃ alkyl, but the present invention is not limited thereto. For example, the alkyl may be methyl, ethyl, n-propyl, isopropyl, n-butyl, isobutyl, secondary butyl (sec-butyl), n-pentyl, isopentyl, neopentyl, isoamyl, n-hexyl, 3-methylhexyl, 2,2-dimethylpentyl, 2,3-dimethylpentyl, or n-heptyl. In the present invention, the alkyl may preferably be an aminoalkyl or a haloalkyl. The alkylamino group refers to an alkyl group substituted with one or more N atoms, and the haloalkyl refers to an alkyl group substituted with one or more halogen atoms.

In the present invention, the alkoxy refers to an alkyl single-bonded to an oxygen atom (—O—R). In the present invention, the alkoxy group may be a C₁-C₁₀, C₁-C₈, C₁-C₆, C₁-C₈, C₁-C₃, or C₁-C₂ alkoxy, but the present invention is not limited thereto. For example, the alkoxy may refer to methoxy, ethoxy, phenoxy, butoxy, or the like.

In the present invention, the alkenyl refers to a straight, branched, or cyclic hydrocarbon radical containing 2 to 20 carbon atoms and one or more carbon-to-carbon double bonds. In the present invention, the alkenyl group may be a C₂-C₁₀, C₂-C₈, C₂-C₆, C₂-C₅, or C₂-C₃ alkenyl group, but the present invention is not limited thereto. Examples of alkenyl radicals include ethenyl, propenyl, allyl, butenyl, and 4-methylbutenyl.

In the present invention, the alkynyl refers to a straight, branched, or cyclic hydrocarbon radical containing 2 to 20 carbon atoms and one or more carbon-to-carbon triple bonds. In the present invention, the alkynyl group may be a C₂-C₁₀, C₂-C₈, C₂-C₆, C₂-C₅, or C₂-C₃ alkynyl group, but the present invention is not limited thereto. For example, the alkynyl group may include propargyl, butynyl, and the like.

In the present invention, the aryl refers to an aromatic system including one or more rings used alone or in combination thereof, and also includes a group in which an aromatic ring is fused to one or more carbon rings. In the present invention, the aryl may be a C₃-C₂₀, C₃-C₁₅, C₃-C₁₂, C₃-C₁₀, C₃-C₈, C₃-C₆, C₃-C₅, C₆-C₂₀, C₆-C₈₅, C₆-C₁₆, C₆-C₁₄, C₆-C₁₂, or C₆-C₁₀ aryl, but the present invention is not limited thereto. For example, the aryl may refer to phenyl, benzyl, naphthyl, or tetrahydronaphthyl, but the present invention is not limited thereto.

In the present invention, the term “substituted” in the “substituted or unsubstituted” means that when one or more hydrogen atoms in an organic compound are replaced with another atomic group to form a derivative, it is introduced in place of a hydrogen atom, and the substituent refers to the introduced atomic group. In the present invention, an alkyl, an alkoxy, a cycloalkyl, a heterocycloalkyl aryl, and a heteroaryl may each independently have one or more hydrogen atoms replaced with another atomic group. In the present invention, the term “substitution” includes single substitution, double substitution, triple substitution, quadruple substitution, and the like.

In the present invention, the term “isomer” refers to a compound having the same molecular formula but different connection patterns or spatial arrangements of constituent atoms within the molecule. Isomers include, for example, structural isomers and stereoisomers. The stereoisomers may be diastereomers or enantiomers. Enantiomers refer to isomers that do not overlap their mirror images, like the relationship between left and right hands, and are also called optical isomers. Enantiomers are distinguished as R (Rectus: clockwise) and S (Sinister: counterclockwise) when four or more substituents are different at the chiral center carbon. Diastereoisomers are stereoisomers that do not have a mirror image relationship, and may be divided into cis-trans isomers due to the different spatial arrangements of atoms.

In the present invention, the term “pharmaceutically acceptable salt” includes salts derived from pharmaceutically acceptable inorganic acids, organic acids, or bases.

As used in this specification, the term “pharmaceutically acceptable” means a compound or composition that is suitable for use in contact with the tissues of a subject (e.g., a human) without excessive toxicity, irritation, allergic responses, or other problems or complications and with a reasonable benefit/risk ratio, and falls within the scope of sound medical judgment.

Examples of suitable acids include hydrochloric acid, bromic acid, sulfuric acid, nitric acid, perchloric acid, hydroiodic acid, fumaric acid, maleic acid, phosphoric acid, glycolic acid, lactic acid, salicylic acid, succinic acid, toluene-p-sulfonic acid, tartaric acid, (+)-L-tartaric acid, acetic acid, trichloroacetic or trifluoroacetic acid, 2,2-dichloroacetic acid, acylated amino acids, adipic acid, alginic acid, ascorbic acid, L-aspartic acid, 4-acetamidobenzoic acid, (+)-camphoric acid, camphorsulfonic acid, (+)-(1S)-camphorsulfonic acid, capric acid, caproic acid, caprylic acid, cinnamic acid, cyclamic acid, dodecylsulfuric acid, ethane-1,2-disulfonic acid, ethanesulfonic acid, 2-hydroxyethanesulfonic acid, galactaric acid, gentisic acid, glucoheptanoic acid, D-gluconic acid, D-glucuronic acid, L-glutamic acid, α-oxoglutaric acid, hippuric acid, (+)-L-lactic acid, (+−)-DL-lactic acid, lactobionic acid, (−)-L-malic acid, (+)-DL-mandelic acid, citric acid, methanesulfonic acid, formic acid, benzoic acid, malonic acid, gluconic acid, naphthalene-2-sulfonic acid, naphthalene-1,5-disulfonic acid, benzenesulfonic acid, 1-hydroxy-2-naphthoic acid, nicotinic acid, oleic acid, orotic acid, oxalic acid, palmitic acid, pamoic acid, L-pyroglutamic acid, salicylic acid, 4-amino-salicylic acid, sebacic acid, stearic acid, tannic acid, thiocyanic acid, camsylic acid, undecylic acid, and the like. Acid addition salts may be prepared by conventional methods, for example, by dissolving the compound in an excess of an aqueous acid solution and precipitating the salt using a water-miscible organic solvent such as methanol, ethanol, acetone, or acetonitrile. Also, the acid addition salts may be prepared by heating equimolar amounts of the compound and an acid or alcohol in water, and then evaporating the mixture to dryness, or subjecting the precipitated salt to suction filtration.

Salts derived from suitable bases may include alkali metals such as sodium, potassium, and the like; alkaline earth metals such as magnesium and the like; ammonium, and the like, but the present invention is not limited thereto. The alkali metal or alkaline earth metal salt may be obtained, for example, by dissolving the compound in an excess of an alkali metal hydroxide or alkaline earth metal hydroxide solution, filtering out the undissolved compound salt, and then evaporating and drying the filtrate. In this case, it is particularly suitable for pharmaceutical purposes to produce a sodium, potassium or calcium salt as the metal salt. Also, a silver salt corresponding to the metal salt may be obtained by reacting an alkali metal or alkaline earth metal salt with a suitable silver salt (e.g., silver nitrate).

A range of the compounds of the present invention may include not only pharmaceutically acceptable salts, but also all isomers, hydrates and solvates that may be prepared by conventional methods.

The compound according to the present invention, an isomer thereof, or a pharmaceutically acceptable salt thereof may be used for the prevention, amelioration, and/or treatment of metabolic diseases.

The compound may be preferably represented by Chemical Formula I-1, I-2, or I-3, and more preferably may be any one of compounds 1 to 18 shown in Table 1 of this specification, but the present invention is not limited thereto.

In this specification, the term metabolic disease refers to a disease in which various symptoms, such as obesity, diabetes, hypertension, hyperlipidemia, arteriosclerosis, coronary syndrome, non-alcoholic fatty liver, and the like, develop in combination due to chronic metabolic disorders, and generally includes the complications caused by these symptoms. The complications caused by these symptoms include diabetic complications such as diabetic retinopathy, diabetic nephropathy, diabetic foot disease, diabetic neuropathy, and the like, cardiovascular diseases such as hyperlipidemia, stroke, angina pectoris, myocardial infarction, peripheral vascular disease, and the like. However, all diseases caused by metabolic diseases may be included without limitation.

In this specification, atherosclerosis is caused by the formation of plaques in which multiple layers of fat accumulate on the arterial vessel wall, and these plaques contain cholesterol lumps, immune cells, vascular smooth muscle cells, and secretions from these cells. Atherosclerosis progresses in stages. At the beginning, endothelial cells express adhesion molecules due to various environmental factors, which results in increased adhesion of monocytes, and monocytes move to the intimal layer and differentiate into macrophages. Next, the macrophages develop into foam cells through oxLDL uptake, and macrophages and T lymphocytes present in the foam cell layer secrete inflammatory cytokines, which induce the migration of vascular smooth muscle cells into the vascular intima. Finally, vascular smooth muscle cells that have moved into the blood vessel thicken the blood vessel wall through replication, secretion of extracellular matrix, and the like to form plaques, thrombi, and the like, thereby causing atherosclerosis.

In this specification, AMPK is a major regulatory factor of energy metabolism, and it is known that its activity increases when Thr172 of the α-subunit is phosphorylated when energy is insufficient. AMPK phosphorylates acetyl-CoA carboxylase (ACC) to inhibit the activity of ACC. ACC is an enzyme that promotes malonyl-CoA synthesis, and malonyl-CoA acts as an inhibitor of carnitine palmitoyltransferase 1 (CPT1), which is a major enzyme of fatty acid oxidation. In this case, an increase in AMPK activity is known to ultimately increase the activity of CPT1. Therefore, AMPK is being actively studied as an important target for diabetes treatment.

The content of the compound in the composition of the present invention may be appropriately adjusted depending on the symptoms of a disease, the degree of progression of the symptoms, the condition of the patient, and the like. For example, the content of the compound may range from 0.0001 to 99.9% by weight, or from 0.001 to 50% by weight based on the total weight of the composition, but the present invention is not limited thereto. The content ratio is a value based on the dry amount from which the solvent is removed.

The pharmaceutical composition according to the present invention may further include suitable carriers, excipients and diluents commonly used in the preparation of pharmaceutical compositions. For example, the excipient may include one or more selected from the group consisting of a diluent, a binder, a disintegrant, a lubricant, an adsorbent, a moisturizer, a film-coating material, and a controlled-release additive.

The pharmaceutical compositions according to the present invention may be separately formulated and used in the form of external preparations, such as powders, granules, sustained-release granules, enteric granules, solutions, eye drops, elixirs, emulsions, suspensions, spirits, troches, perfumes, limonades, tablets, sustained-release tablets, enteric tablets, sublingual tablets, hard capsules, soft capsules, sustained-release capsules, enteric capsules, pills, tinctures, soft extracts, dry extracts, fluid extracts, injections, capsules, perfusates, plasters, lotions, pastes, sprays, inhalants, patches, sterile injection solutions, aerosols, or the like, according to conventional methods. Also, the external preparations may have a formulation such as a cream, a gel, a patch, a spray, an ointment, a plaster, a lotion, a liniment, a paste, a cataplasma, or the like.

The carriers, excipients, and diluents that may be included in the pharmaceutical composition according to the present invention include lactose, dextrose, sucrose, oligosaccharides, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil.

When formulated, the pharmaceutical composition is prepared using commonly used diluents or excipients such as fillers, extenders, binders, wetting agents, disintegrants, surfactants, and the like.

As the additives for tablets, powders, granules, capsules, pills, and troches according to the present invention, excipients such as corn starch, potato starch, wheat starch, lactose, sucrose, glucose, fructose, di-mannitol, precipitated calcium carbonate, synthetic aluminum silicate, calcium monohydrogen phosphate, calcium sulfate, sodium chloride, sodium hydrogen carbonate, purified lanolin, microcrystalline cellulose, dextrin, sodium alginate, methyl cellulose, sodium carboxymethyl cellulose, kaolin, urea, colloidal silica gel, hydroxypropyl starch, hydroxypropylmethyl cellulose (HPMC) 1928, HPMC 2208, HPMC 2906, HPMC 2910, propylene glycol, casein, calcium lactate, Primojel, and the like; binders such as gelatin, gum arabic, ethanol, agar powder, cellulose acetate phthalate, carboxymethyl cellulose, calcium carboxymethyl cellulose, glucose, purified water, sodium caseinate, glycerin, stearic acid, sodium carboxymethyl cellulose, sodium methyl cellulose, methyl cellulose, microcrystalline cellulose, dextrin, hydroxycellulose, hydroxypropyl starch, hydroxymethyl cellulose, purified shellac, gelatinized starch, hydroxypropyl cellulose, hydroxypropyl methyl cellulose, polyvinyl alcohol, polyvinyl pyrrolidone, and the like may be used. Also, disintegrants such as hydroxypropyl methyl cellulose, corn starch, agar powder, methyl cellulose, bentonite, hydroxypropyl starch, sodium carboxymethyl cellulose, sodium alginate, calcium carboxymethyl cellulose, calcium citrate, sodium lauryl sulfate, silicic anhydrides, 1-hydroxy propyl cellulose, dextran, ion exchange resins, polyvinyl acetate, formaldehyde-treated casein and gelatin, alginic acid, amylose, guar gum, sodium hydrogen carbonate, polyvinyl pyrrolidone, calcium phosphate, gelatinized starch, gum arabic, amylopectin, pectin, sodium polyphosphate, ethyl cellulose, sucrose, magnesium aluminum silicate, a di-sorbitol solution, light silicic anhydrides, and the like; lubricants such as calcium stearate, magnesium stearate, stearic acid, hydrogenated vegetable oil, talc, lycopodium, kaolin, petroleum jelly, sodium stearate, cacao butter, sodium salicylate, magnesium salicylate, polyethylene glycol (PEG) 4000, PEG 6000, liquid paraffin, hydrogenated soybean oil (Lubri wax), aluminum stearate, zinc stearate, sodium lauryl sulfate, magnesium oxide, Macrogol, synthetic aluminum silicate, silicic anhydrides, higher fatty acids, higher alcohols, silicone oil, paraffin oil, polyethylene glycol fatty acid ethers, starch, sodium chloride, sodium acetate, sodium oleate, dl-leucine, light silicic anhydrides, and the like may be used.

As the additives for solutions according to the present invention, water, dilute hydrochloric acid, dilute sulfuric acid, sodium citrate, sucrose monostearates, polyoxyethylene sorbitol fatty acid esters (Tween esters), polyoxyethylene monoalkyl ethers, lanolin ethers, lanolin esters, acetic acid, hydrochloric acid, aqueous ammonia, ammonium carbonate, potassium hydroxide, sodium hydroxide, prolamine, polyvinyl pyrrolidone, ethyl cellulose, sodium carboxymethyl cellulose, and the like may be used.

In the syrup according to the present invention, a solution of sucrose, other sugars, or a sweetener may be used. When necessary, aromatics, coloring agents, preservatives, stabilizers, suspending agents, emulsifiers, thickeners, and the like may be used.

Purified water may be used in the emulsions according to the present invention, and emulsifiers, preservatives, stabilizing agents, fragrances, and the like may be used when necessary.

As the suspending agents according to the present invention, suspending agents such as acacia, tragacanth, methyl cellulose, carboxymethyl cellulose, sodium carboxymethyl cellulose, microcrystalline cellulose, sodium alginate, hydroxypropyl methyl cellulose (HPMC), HPMC 1828, HPMC 2906, HPMC 2910, and the like may be used. When necessary, surfactants, preservatives, stabilizers, coloring agents, and fragrances may be used.

The injections according to the present invention may include solvents such as distilled water for injection, 0.9% sodium chloride injection, Ringer's solution, dextrose injection, dextrose+sodium chloride injection, PEG, lactated Ringer's solution, ethanol, propylene glycol, non-volatile sesame oil, cottonseed oil, peanut oil, soybean oil, corn oil, ethyl oleate, isopropyl myristate, benzene benzoate, and the like; solubilizing aids such as sodium benzoate, sodium salicylate, sodium acetate, urea, urethane, monoethylacetamide, butazolidine, propylene glycol, Tweens, nicotinamide, hexamine, dimethylacetamide, and the like; buffers such as weak acids and salts thereof (acetic acid and sodium acetate), weak bases and salts thereof (ammonia and ammonium acetate), organic compounds, proteins, albumin, peptones, gums, and the like; isotonic agents such as sodium chloride; stabilizers such as sodium bisulfite (NaHSO₃), carbon dioxide gas, sodium metabisulfite (Na₂S₂O₅), sodium sulfite (Na₂SO₃), nitrogen gas (N₂), ethylene diamine tetraacetic acid, and the like; antioxidants such as 0.1% sodium bisulfide, sodium formaldehyde sulfoxylate, thiourea, ethylene diamine disodium tetraacetate, acetone sodium bisulfite, and the like; analgesics such as benzyl alcohol, chlorobutanol, procaine hydrochloride, glucose, calcium gluconate, and the like; and suspending agents such as CMC sodium, sodium alginate, Tween 80, aluminum monostearate, and the like.

In the suppositories according to the present invention, bases such as cacao butter, lanolin, Witepsol, polyethylene glycol, glycerogelatin, methyl cellulose, carboxymethyl cellulose, a mixture of stearic acid and oleic acid, Subanal, cottonseed oil, peanut oil, palm oil, cacao butter+cholesterol, lecithin, Lanette Wax, glycerol monostearate, Tween or Span, Imhausen, monolene (propylene glycol monostearate), glycerin, Adeps solidus, Buytyrum Tego-G, Cebes Pharma 16, Hexalide Base 95, Cotomar, Hydrokote SP, S-70-XXA, 5-70-XX75 (S-70-XX95), Hydrokote 25, Hydrokote 711, Idropostal, Massa estrarium (A, AS, B, C, D, E, I, T), Massa-MF, Marsupol, Marsupol-15, Neosupostal-N, Paramound-B, Suposiro (OSI, OSIX, A, B, C, D, H, L), suppository type IV (AB, B, A, BC, BBG, E, BGF, C, D, 299), Supostal (N, Es), Wecobi (W, R, S, M, Fs), Tegester triglyceride bases (TG-95, MA, 57), and the like may be used.

Solid formulations for oral administration include tablets, pills, powders, granules, capsules, and the like, and such solid formulations may be prepared by mixing an extract with at least one excipient such as starch, calcium carbonate, sucrose or lactose, gelatin, and the like. In addition to simple excipients, lubricants such as magnesium stearate and talc may also be used.

Liquid formulations for oral administration include suspensions, internal solutions, emulsions, syrups, and the like. In this case, various excipients such as wetting agents, sweeteners, flavoring agents, preservatives, and the like may be included in addition to commonly used simple diluents such as water, liquid paraffin, and the like. Formulations for parenteral administration include sterile aqueous solutions, non-aqueous solvents, suspensions, emulsions, freeze-dried formulations, and suppositories. Propylene glycol, polyethylene glycol, vegetable oils such as olive oil, injectable esters such as ethyl oleate, and the like may be used as the non-aqueous solvents and suspending agents.

The pharmaceutical composition according to the present invention may be administered in a pharmaceutically effective amount. In the present invention, the “pharmaceutically effective amount” refers to an amount sufficient to treat a disease with a reasonable benefit/risk ratio applicable to medical treatment, and a level of the effective amount may be determined according to the type of a patient's disease, the severity of the disease, the activity of a drug, the sensitivity to the drug, the time of administration, the route of administration, and the excretion rate, the duration of treatment, factors including drugs used concurrently, and other factors well known in the medical field.

The pharmaceutical composition according to the present invention may be administered as an individual therapeutic agent or in combination with other therapeutic agents, and may be administered sequentially or concurrently with a conventional therapeutic agent and may be administered once or multiple times. It is important to administer the pharmaceutical composition according to the present invention in an amount sufficient to obtain the maximum effect with the minimum amount without side effects in consideration of all the above-mentioned factors, and such an amount may be easily determined by a person having ordinary skill in the technical field to which the present invention pertains.

The pharmaceutical composition according to the present invention may be administered to a subject through various routes of administration. All modes of administration may be contemplated, and may, for example, include oral administration, subcutaneous injection, intraperitoneal administration, intravenous injection, intramuscular injection, injection into the paraspinal space (intrathecal), sublingual administration, buccal administration, rectal insertion, vaginal insertion, intraocular administration, auricular administration, intranasal administration, inhalation, spraying through the mouth or nose, intradermal administration, transdermal administration, and the like.

The pharmaceutical composition of the present invention may be determined according to the type of drug as an active ingredient together with various related factors such as the type of disease to be treated, the route of administration, the age, gender, and weight of a patient, the severity of a disease, and the like. Specifically, the effective amount of the composition according to the present invention may vary depending on the age, weight, and gender of a patient. In general, the pharmaceutical composition may be administered daily, every other day, or 1 to 3 times a day at a dose of 0.001 to 150 mg, more preferably 0.01 to 100 mg per 1 kg of body weight. However, since the dosage may increase or decrease depending on the route of administration, the severity of obesity, the gender, weight, and age of a patent, and the like, the dosage is not intended to limit the scope of the present invention in any way.

In the present invention, the “subject” refers to a subject requiring treatment for a disease. More specifically, the subject may be a mammal such as a human or non-human primate, a mouse, a rat, a dog, a cat, a horse, or cattle, but the present invention is not limited thereto.

In the present invention, “administration” refers to any action of providing a predetermined composition of the present invention to a subject using any suitable method.

In the present invention, “prevention” refers to any action of inhibiting or delaying the onset of a target disease, “treatment” refers to any action of improving or beneficially changing a target disease and metabolic abnormality symptoms thereof by administering the pharmaceutical composition according to the present invention, and “amelioration” refers to any action of reducing parameters related to the target disease, for example, the degree of symptoms thereof, by administering the composition according to the present invention.

Also, the present invention provides a kit for preventing or treating a metabolic disease, which includes the compound according to the present invention, an isomer thereof, or a pharmaceutically acceptable salt thereof as an active ingredient. In the present invention, the term “kit” refers to a combination of materials or devices for preventing or treating a metabolic disease using the compound according to the present invention, and has no limitations on its specific form. The kit according to the present invention may include not only the compound according to the present invention for preventing and/or treating a metabolic disease, but also one or more kinds of other component compositions, solutions, devices, or instructions suitable for the prevention, amelioration, or treatment of diseases, the instructions presenting a method of preparing the compound according to the present invention or a suitable method of using the same.

Also, the present invention provides a food composition for preventing or ameliorating a metabolic disease, which includes the compound according to the present invention, an isomer thereof, or a food-wise acceptable salt thereof as an effective ingredient. The food composition includes a health functional food composition.

When the compound of the present invention is used as a food additive, the compound may be added as is, or used together with other foods or food ingredients. In this case, the compound of the present invention may be used appropriately according to conventional methods. The mixing amount of the active ingredient may be appropriately determined according to the purpose of use (prevention, health or therapeutic treatment). In general, when foods or beverages are prepared, the compound of the present invention may be added in an amount of 15% by weight or less, or 10% by weight or less based on the total weight of the raw material. However, in the case of long-term intake for the purpose of health and hygiene or for the purpose of health control, the amount may be less than the above range. Since the active ingredient has no problems in terms of safety, the active ingredient may be used in an amount greater than the above range.

There is no particular limitation on the type of food. Examples of foods to which the above substance may be added may include meat, sausage, bread, chocolate, candies, snacks, confectioneries, pizza, ramen, other noodles, gum, dairy products including ice cream, various soups, beverages, tea, drinks, alcoholic beverages, vitamin complexes, and the like, and include all health functional foods in the usual sense.

A health beverage composition according to the present invention may contain various flavoring agents, natural carbohydrates, and the like as additional components, as in conventional beverages. The above-described natural carbohydrates include monosaccharides such as glucose and fructose, disaccharides such as maltose and sucrose, polysaccharides such as dextrin and cyclodextrin, and sugar alcohols such as xylitol, sorbitol, erythritol, and the like. As sweeteners, natural sweeteners such as thaumatin and stevia extracts, synthetic sweeteners such as saccharin and aspartame, and the like may be used. The proportion of the natural carbohydrates per 100 mL of the composition of the present invention may generally range from approximately 0.01 to 0.20 g, or approximately 0.04 to 0.10 g.

In addition to the above-described substances, the composition of the present invention may contain various nutrients, vitamins, electrolytes, flavors, colorants, pectic acid and salts thereof, alginic acid and salts thereof, organic acids, protective colloidal thickeners, pH adjusters, stabilizing agents, preservatives, glycerin, alcohol, carbonating agents used for carbonated beverages, and the like. In addition, the composition of the present invention may contain fruit flesh for preparing natural fruit juice, fruit juice beverages, and vegetable beverages. These ingredients may be used individually or in combination. The proportion of these additives is not highly critical, but may generally be selected in a range of 0.01 to 0.20 parts by weight based on 100 parts by weight of the composition of the present invention.

In this specification, the term “health functional food” is the same term as a food for special health use (FoSHU), and refers to a processed food with high medicinal and medical effects to efficiently exhibit bio-regulatory functions in addition to nutrient supply. In this case, the food may be prepared in various forms such as tablets, capsules, powders, granules, liquids, pills, and the like in order to obtain useful effects for preventing or ameliorating a metabolic disease.

The health functional food of the present invention may be prepared by a method commonly used in the art, and may be prepared by adding raw materials and components commonly added in the art during the preparation. Also, unlike general drugs, the health functional food of the present invention has an advantage in that it has no side effects that may occur when taking a drug for a long time because food is used as a raw material, and may have excellent portability.

Throughout the specification of the present invention, when any certain part is said to “include” any component, this means that it may further include other components, rather than excluding other components unless otherwise stated. In addition, the terms “approximately,” “substantially,” the like used throughout the specification of the present invention are used at, or in close proximity to, numerical values when manufacturing and material tolerances inherent in the indicated meanings are provided. In this case, precise or absolute figures are used to aid in understanding the present invention and to assist in the prevention of unfair use by unscrupulous infringers.

Throughout the specification of the present invention, the term “combination thereof” included in a Markush type expression refers to a mixture or combination of one or more selected from the group consisting of components disclosed in the Markush type expression, and thus means that the mixture or combination include one or more selected from the group consisting of the components.

### MODE FOR INVENTION

Hereinafter, preferred embodiments of the present invention are presented in order to aid in understanding the present invention. However, it should be understood that the following examples are given by way of illustration only to more easily understand the present invention, and are not intended to limit the present invention.

### EXAMPLES

### Example 1: Preparation of DMC Derivatives According to the Present Invention

Among the novel derivatives of 2′,4′-dihydroxy-6′-methoxy-3′,5′-dimethylchalcone (DMC) according to the present invention, Compounds 1 to 7 and 8 to 14 were synthesized according to the process shown in the following Scheme 1.

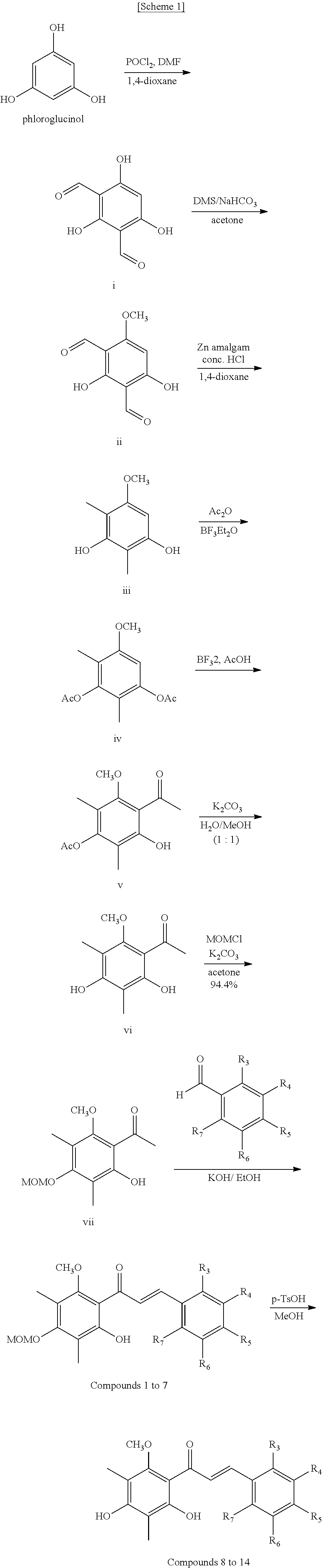

### Example 1-1: Synthesis of 2,4-diformylphloroglucinol (Compound i)

Phosphorus oxychloride (59.3 mL, 0.634 mol) was added dropwise to DMF (49.1 mL, 0.634 mol) while stirring at 0° C. When the addition was completed, the mixture was stirred vigorously at 25° C. for 30 minutes. The resulting yellow viscous liquid (Vilsmeier reagent) was added dropwise to a solution of dry phloroglucinol (40.0 g, 0.317 mol) in 1,4-dioxane (200 mL) while stirring at 0° C. After vigorous stirring at 25° C. for 4 hours, the resulting yellow amorphous solid was transferred to a 2 L round-bottom flask using water (1.5 L), and stirred vigorously at 25° C. for 3 hours. The precipitated yellow solid was filtered, washed with water, and dried in a vacuum oven at 30° C. for 12 hours to obtain Compound i (56.8 g, 0.312 mol). The crude product had sufficiently high purity for use in the subsequent steps without further purification: m.p. 221-224° C.; TLC R_{f}=0.21 (n-hexane/acetone=1:2); IR νₘₐₓ (cm⁻¹) 2888, 1599, 1503, 1439, 1393, 1254, 1187; ¹H NMR (DMSO-d₆, 300 MHz) δ12.52 (br s, 2H, —OH), 10.01 (s, 2H, —CHO), 5.90 (s, 1H, Ar—H), one hydroxyl proton was not observable, presumably owing to rapid proton exchange; ¹³C NMR (DMSO-d₆, 150 MHz) δ191.4 (2C), 169.4 (2C), 169.0 (1C), 103.8 (2C), 94.1 (1C).

### Example 1-2: Synthesis of 1,3-dihydroxy-2,4-diformyl-5-methoxybenzene (Compound ii)

A solution of Compound i (9.00 g, 49.4 mmol) in dry acetone (500 mL) was stirred for 10 minutes under a N₂ atmosphere, and dimethyl sulfate (5.16 mL, 54.4 mmol) and NaHCO₃ (1.66 g, 19.8 mmol) were then added. Also, NaHCO₃ (1.66 g, 19.8 mmol) was further added twice at 12 hour intervals, and the mixture was stirred at 42° C. for 8 days. The reaction mixture was cooled to 25° C., and extracted with EtOAc (100 mL). The organic layer was washed with an aqueous 1% HCl solution (100 mL), water (3×200 mL), and an aqueous saturated NaCl solution (200 mL), and then dried over MgSO₄. The organic solvent was evaporated under reduced pressure, and the crude product was purified by column chromatography (n-hexane/acetone=20:1) to obtain Compound ii (6.51 g, 33.2 mmol) as a white solid: m.p. 139-140° C.; TLC R_{f}=0.65 (n-hexane/acetone=3:2); IR νₘₐₓ (cm⁻¹) 2898, 1614, 1594, 1189, 1081; ¹H NMR (CDCl₃, 300 MHz,) δ 13.64 (s, 1H, —OH), 13.09 (s, 1H, —OH), 10.18 (s, 1H, —CHO), 10.05 (s, 1H, —CHO), 5.92 (s, 1H, Ar—H), 3.95 (s, 3H, —OCH₃); ¹³C NMR (DMSO-d₆, 150 MHz) δ 191.9 (1C), 191.5 (1C), 171.2 (1C), 168.9 (1C), 168.9 (1C), 104.6 (1C), 104.5 (1C), 92.0 (1C), 57.5 (1C).

### Example 1-3: Synthesis of 1,3-dihydroxy-2,4-dimethyl-5-methoxybenzene (Compound iii)

Zinc powder (30.0 g) was added to an aqueous 1% HCl solution (300 mL), and activated while stirring for an hour. Mercury (II) chloride (0.900 g) was added to an aqueous 3% HCl solution (150 mL), and the mixture was then stirred vigorously at 25° C. for 4 hours. The resulting fluffy solid was filtered, washed with 1,4-dioxane, and added to a stirred solution of Compound ii (3.00 g, 15.3 mmol) in 1,4-dioxane (200 mL). The reaction mixture was stirred at 25° C. for 20 minutes, and then cooled to 0° C. Thereafter, an aqueous 36% HCl solution (12 mL) was slowly added, and the mixture was stirred at 0° C. for 30 minutes. The reaction mixture was filtered, diluted with water (200 mL), and extracted with EtOAc (3×100 mL). The combined organic layers were washed with an aqueous saturated NaCl solution (200 mL), and dried over MgSO₄. The organic solvent was evaporated under reduced pressure, and the crude product was purified by column chromatography (n-hexane/acetone=30:1) to obtain Compound iii (2.44 g, 14.5 mmol) as a white solid: m.p. 95-96° C.; TLC R_{f}=0.50 (n-hexane/acetone=3:2); IR νₘₐₓ (cm⁻¹) 3376, 2921, 2852, 1615, 1505, 1454, 1330, 1276, 1208, 1113, 1089; ¹H NMR (DMSO-d₆, 300 MHz) δ 8.83 (s, 1H, —OH), 7.95 (s, 1H, —OH), 6.01 (s, 1H, Ar—H), 3.63 (s, 3H, —OCH₃), 1.91 (s, 3H, —CH₃), 1.90 (s, 3H, —CH₃); ¹³C NMR (DMSO-d₆, 150 MHz) δ 155.9 (1C), 154.4 (1C), 154.0 (1C), 103.7 (1C), 103.2 (1C), 91.5 (1C), 55.5 (1C), 9.2 (1C), 9.0 (1C).

### Example 1-4: Synthesis of 1,3-diacetoxy-2,4-dimethyl-5-methoxybenzene (Compound iv)

BF₃Et₂O (4.85 mL, 39.3 mmol) was added to a solution of Compound iii (5.50 g, 32.7 mmol) in acetic anhydride (30.9 mL, 327 mmol) at 0° C. under a N₂ atmosphere. After stirring at 25° C. for an hour, the reaction mixture was diluted with EtOAc (100 mL), washed with an aqueous 1% HCl solution (100 mL), water (3×200 mL), and an aqueous saturated NaCl solution (200 mL), and then dried over MgSO₄. The organic solvent was evaporated under reduced pressure, and the crude product was purified by column chromatography (n-hexane/acetone=300:1) to obtain Compound iv (7.38 g, 29.3 mmol) as a clear oil: TLC R_{f}=0.61 (n-hexane/acetone=3:2); IR νₘₐₓ (cm⁻¹) 2935, 1756, 1368, 1189, 1116, 1009, 1074; ¹H NMR (DMSO-d₆, 600 MHz) δ 6.69 (s, 1H, Ar—H), 3.75 (s, 3H, —OCH₃), 2.34 (s, 3H, —OCOCH₃), 2.29 (s, 3H, —OCOCH₃), 1.91 (s, 3H, —CH₃), 1.81 (s, 3H, —CH₃); ¹³C NMR (DMSO-d₆, 150 MHz) δ168.8 (1C), 168.3 (1C), 155.7 (1C), 148.3 (1C), 147.5 (1C), 116.0 (1C), 114.6 (1C), 103.1 (1C), 55.8 (1C), 20.5 (1C), 20.1 (1C), 9.3 (1C), 9.1 (1C); Anal. Calcd for C₁₃H₁₆O₅: C, 61.90; H, 6.39. Found: C, 62.19; H, 6.32.

### Example 1-5: Synthesis of 2-hydroxy-4-acetoxy-6-methoxy-3,5-dimethylacetophenone (Compound v)

BF₃2AcOH (15.4 mL, 111.1 mmol) was added to Compound iv (7.00 g, 27.7 mmol), and the mixture was refluxed for 4 hours under a N₂ atmosphere. The reaction mixture was cooled to 25° C., diluted with EtOAc (100 mL), washed with an aqueous 1% HCl solution (100 mL), water (3×200 mL), and an aqueous saturated NaCl solution (200 mL), and then dried over MgSO₄. The organic solvent was evaporated under reduced pressure, and the crude product was purified by silica gel column chromatography (n-hexane/acetone=50:1) to obtain Compound v (4.57 g, 18.1 mmol) as a white solid: m.p. 79-80° C.; TLC R_{f}=0.71 (n-hexane/acetone=3:2); IR νₘₐₓ (cm⁻¹) 2922, 1743, 1615, 1585, 1406, 1386, 1361, 1316, 1283, 1219, 1169; ¹H NMR (DMSO-d₆, 300 MHz) δ 12.28 (s, 1H, —OH), 3.70 (s, 3H, —OCH₃), 2.65 (s, 3H, —COCH₃), 2.37 (s, 3H, —OCOCH₃), 1.96 (s, 3H, —CH₃), 1.91 (s, 3H, —CH₃); ¹³C NMR (DMSO-d₆, 150 MHz) δ 204.3 (1C), 168.0 (1C), 157.4 (1C), 157.1 (1C), 152.8 (1C), 115.0 (1C), 114.9 (1C), 114.7 (1C), 61.9 (1C), 31.8 (1C), 20.1 (1C), 9.2 (1C), 8.9 (1C); Anal. Calcd for C₁₃H₁₆O₅: C, 61.90; H, 6.39. Found: C, 62.28; H, 6.46.

### Example 1-6: Synthesis of 2,4-dihydroxy-6-methoxy-3,5-dimethylacetophenone (Compound vi)

K₂CO₃ (9.96 g, 72.1 mmol) was added to a solution of Compound v (4.54 g, 18.0 mmol) in MeOH/water (1:1, 30 mL). The mixture was sonicated for 20 minutes, and stirred overnight. The reaction mixture was diluted with EtOAc (100 mL), washed with an aqueous 1% HCl solution (100 mL), water (3×200 mL), and an aqueous saturated NaCl solution (200 mL), and then dried over MgSO₄. The organic solvent was evaporated under reduced pressure, and the crude product was purified by column chromatography (n-hexane/acetone=250:1) to give Compound vi as a pale yellow solid. The product was recrystallized from DCM to obtain a yellow crystalline solid (3.65 g, 17.4 mmol).

### Example 1-7: Synthesis of 2-hydroxy-4-methoxymethoxy-6-methoxy-3,5-dimethylacetophenone (Compound vii)

Chloromethyl methyl ether (0.411 mL, 5.42 mmol) was added to a suspension of Compound vi (0.950 g, 4.52 mmol) and K₂CO₃ (0.749 g, 5.42 mmol) in dry acetone (40 mL) under a N₂ atmosphere. The solution was refluxed for an hour, and then cooled to 25° C. The mixture was diluted with EtOAc (100 mL), washed with an aqueous 1% HCl solution (100 mL), water (3×200 mL), and an aqueous saturated NaCl solution (200 mL), and the dried over MgSO₄. The organic solvent was evaporated under reduced pressure, and the crude product was purified by column chromatography (n-hexane/acetone=50:1) to obtain Compound vii (1.09 g, 4.27 mmol) as a pale yellow solid: m.p. 59-60° C.; TLC R_{f}=0.68 (n-hexane/acetone=1:1); IR νₘₐₓ (cm⁻¹) 2953, 2922, 2852, 1602, 1454, 1410, 1356, 1317, 1268, 1219, 1173; ¹H NMR (DMSO-d₆, 300 MHz) δ 12.80 (br s, 1H, —OH), 5.00 (s, 2H, —CH₂-0), 3.71 (s, 3H, —OCH₃), 3.51 (s, 3H, —OCH₃), 2.65 (s, 3H, —COCH₃), 2.10 (s, 3H, —CH₃), 2.04 (s, 3H, —CH₃); ¹³C NMR (DMSO-d₆, 150 MHz) δ 204.3 (1C), 160.8 (1C), 159.1 (1C), 158.2 (1C), 115.3 (1C), 114.7 (1C), 112.4 (1C), 99.0 (1C), 61.5 (1C), 57.1 (1C), 31.6 (1C), 9.8 (1C), 9.2 (1C).

### Example 1-8: General Synthesis Process of MOM-DMC Derivatives (Compounds 1 to 7)

Benzaldehyde was added to a solution of Compound vii and KOH in EtOH. The reaction mixture was stirred at 25° C. for 7 days. The mixture was diluted with EtOAc (100 mL), washed with an aqueous 1% HCl solution (100 mL), water (3×200 mL), and an aqueous saturated NaCl solution (200 mL), and then dried over MgSO₄. The organic solvent was evaporated under reduced pressure, and the crude product was purified by column chromatography (n-hexane/acetone=500:1). The product was further purified by recrystallization from an appropriate solvent.

### Example 1-9: General synthesis process of 2′,4′-dihydroxy-6′-methoxy-3′,5′-dimethylchalcone derivatives (Compound 8 to 14)

p-Toluenesulfonic acid was added to a solution of Compounds 1 to 7 in MeOH, and the mixture was stirred overnight at 25° C. The mixture was diluted with EtOAc (100 mL), washed with an aqueous 1% HCl solution (100 mL), water (3×200 mL), and an aqueous saturated NaCl solution (200 mL), and then dried over MgSO₄. The organic solvent was evaporated under reduced pressure to obtain the crude products (Compounds 8 to 14), which were further purified.

### Example 2: Preparation of DMC Derivatives According to the Present Invention-2

Among the novel derivatives of DMC according to the present invention, Compounds 15 to 18 were synthesized according to the process shown in the following Scheme 2. Specific descriptions of Reaction steps 1 to 6 shown in the scheme below are described below.

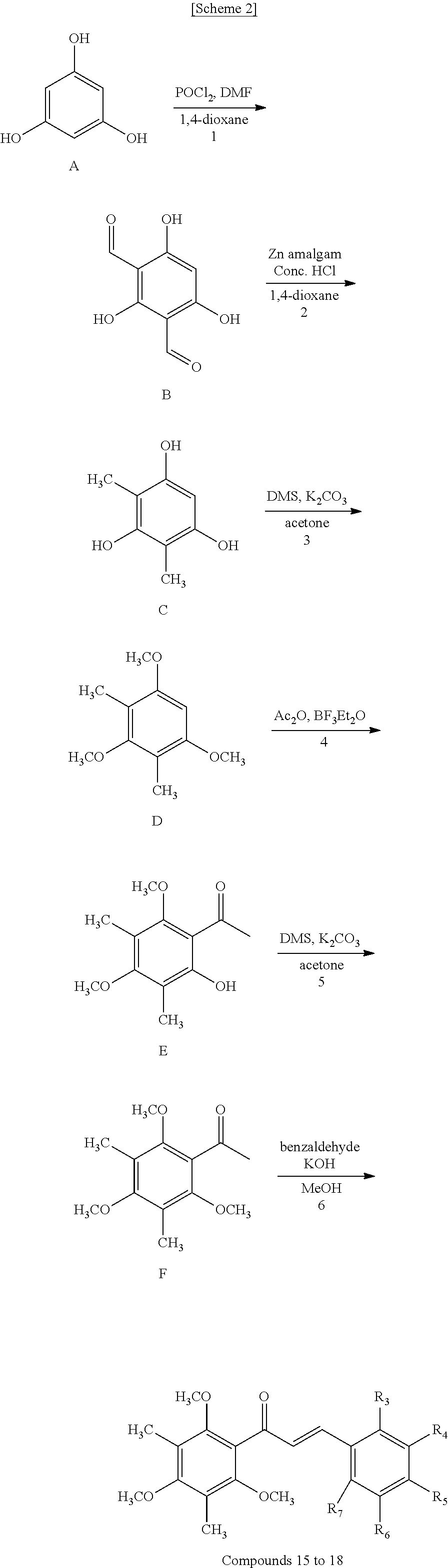

### Reaction Step 1

Dimethylformamide (59.30 mL, 6.34 mol) was added to a 2-neck round-bottom flask, and phosphorus (V) oxychloride (49.12 mL, 6.34 mol) was added dropwise through a dropping funnel at 0° C. while stirring vigorously for 30 minutes. Dry phloroglucinol (40 g, 3.17 mol) of Chemical Formula A was dissolved in 1,4-dioxane (200 mL), and then added dropwise to the previously prepared Vilsmeier reagent at 0° C. while stirring vigorously. After stirring at room temperature for 4 hours or more, a yellow solid was obtained. This compound was transferred to a 2 L round-bottom flask, DI water (1.5 L) was added, and the resulting mixture was stirred vigorously for 3 hours. After stirring, the precipitated yellow solid was filtered and dried in a vacuum oven at 30° C. for 12 hours to obtain a light orange Chemical Formula B (56.84 g, 98.4%).

m.p.=221-224° C.; TLC R_{f}=0.208 (n-hexane:acetone=1:2); IR νₘₐₓ (cm⁻¹) 2887.88, 1598.70, 1503.24, 1438.64, 1393.32, 1253.50, and 1186.97; ¹H NMR (300 MHz, DMSO-d₆) δ12.52 (br s, 2H, —OH), 10.01 (s, 2H, CHO), 5.90 (s, 1H, Ar—H); and ¹³C NMR (150 MHz, DMSO-d₆) δ191.37 (2C), 169.42 (2C), 169.02 (1C), 103.77 (2C), and 94.07 (1C).

### Reaction Step 2

300 mL of an aqueous 1% HCl solution and 30 g of Zn were added to a 1,000 mL beaker, and stirred. Thereafter, 450 mL of 3% HCl and mercury (II) chloride (HgCl₂, 0.9 g) were added, and stirred vigorously at room temperature. The prepared zinc amalgam was washed with water and 1,4-dioxane, and then added to a solution of Chemical Formula B (3 g, 19.46 mmol) and 1,4-dioxane (300 mL) as a solvent, and stirred for 20 minutes. The reaction mixture was cooled to 0° C., and stirred while slowly adding an aqueous 36% HCl solution (12 mL). The mixture was filtered with 200 mL of water, and diluted with 300 mL of ethyl acetate. The organic solvent layer was washed with water and an aqueous saturated NaCl solution, and then dried with MgSO₄. The solvent was removed by distillation under reduced pressure, and the residue was separated using column chromatography (n-hexane:acetone=8:1) to obtain a reddish brown Chemical Formula C (1.717 g, 67.4%).

m.p. 162-163° C.; TLC R_{f}=0.67 (n-hexane:acetone=1:2); IR νₘₐₓ (cm¹) 3527, 3465, 3425, 2921, 2852, 1609, 1457, 1433, 1247, 1150; ¹H NMR (DMSO-d₆, 300 MHz) δ 8.62 (s, 2H, —OH), 7.76 (s, 1H, —OH), 5.92 (s, 1H, Ar—H), 1.86 (s, 6H, —CH₃); ¹³C NMR (DMSO-d₆, 150 MHz) δ 154.1 (2C), 153.2 (2C), 102.6 (1C), 94.6 (1C), 8.7 (2C).

### Reaction Step 3

Chemical Formula C (1.717 g, 11.14 mmol) and potassium carbonate (K₂CO₃, 6.1573 g, 44.55 mmol) were dissolved in dry acetone, dimethyl sulfate (DMS, 4.2154 mL, 44.55 mmol) was added, and the resulting mixture was then refluxed for one day. The mixture was cooled to room temperature, diluted with 300 mL of ethyl acetate, and washed with an aqueous 1% HCl solution. The organic solvent layer was washed with water and an aqueous saturated NaCl solution, and dried with MgSO₄. The solvent was removed by distillation under reduced pressure, and the residue was separated using column chromatography (n-hexane:acetone=300:1) to obtain a white solid Chemical Formula D (2.1550 g, 98.6%).

m.p. 152-155° C.; TLC R_{f}=0.69 (n-hexane:acetone=3:2); IR νₘₐₓ (cm¹) 2926, 1608, 1496, 1464, 1435, 1401, 1321, 1219, 1193, 1127; ¹H NMR (DMSO-d₆, 300 MHz) δ 6.41 (s, 1H, Ar—H), 3.77 (s, 6H, —OCH₃), 3.57 (s, 3H, —OCH₃), 1.98 (s, 6H, —CH₃); ¹³C NMR (DMSO-d₆, 150 MHz) δ 157.2 (1C), 156.3 (2C), 109.9 (2C), 91.8 (1C), 59.7 (1C), 55.5 (2C), 8.5 (2C).

### Reaction Step 4

Acetic anhydride (2.07 mL, 21.96 mmol) was added to Chemical Formula D (2.1550 g, 10.98 mmol). The solution temperature was lowered to 0° C., and boron-trifluoride diethyl etherate (BF₃Et₂O, 2.7582 mL, 21.96 mmol) was added. The resulting mixture was stirred for 3 hours while maintaining the reaction temperature at 90° C. Thereafter, the reaction product was cooled to room temperature, and diluted with 300 mL of ethyl acetate. Then, the organic solvent layer was washed with an aqueous 1% HCl solution, water, and an aqueous saturated NaCl solution, and dried with MgSO₄. The solvent was removed by distillation under reduced pressure, and the residue was separated using column chromatography (n-hexane) to obtain Chemical Formula E (1.8812 g, 76.4%) as a yellow solid.

m.p. 48-50° C.; TLC R_{f}=0.66 (n-hexane:acetone=3:2); IR νₘₐₓ (cm⁻¹) 3440, 2941, 1621, 1454, 1417, 1364, 1318, 1283, 1198, 1171, 1120; ¹H NMR (DMSO-d₆, 300 MHz) δ 12.80 (s, 1H, —OH), 3.71 (s, 3H, —OCH₃), 3.69 (s, 3H, —OCH₃), 2.65 (s, 3H, —COCH₃), 2.09 (s, 3H, —CH₃), 2.03 (s, 3H, —CH₃); ¹³C NMR (DMSO-d₆, 150 MHz) δ203.8 (1C), 163.2 (1C), 158.8 (1C), 156.7 (1C), 115.4 (1C), 114.8 (1C), 113.9 (1C), 61.6 (1C), 59.9 (1C), 31.4 (1C), 8.7 (2C).

### Reaction Step 5

Chemical Formula E (1.8812 g, 8.389 mmol) and potassium carbonate (K₂CO₃, 1.3913 g, 10.06 mmol) were dissolved in dry acetone, dimethyl sulfate (DMS, 0.9519 mL, 10.06 mmol) was added, and the resulting mixture was then refluxed for one day. The reaction mixture was cooled to room temperature, diluted with 300 mL of ethyl acetate, and then washed with an aqueous 1% HCl solution. The organic solvent layer was washed with water and an aqueous saturated NaCl solution, and moisture was removed using MgSO₄. The solvent was removed by distillation under reduced pressure, and the residue was separated using column chromatography (n-hexane:acetone=300:1) to obtain Chemical Formula F (1.933 g, 96.7%) as a white solid.

¹H NMR (300 MHz, DMSO-d₆) δ 3.71 (s, 3H, —OCH₃), 3.69 (s, 6H, —OCH₃), 2.66 (s, 3H, —COCH₃), 2.10 (s, 3H, —CH₃), 2.04 (s, 3H, —CH₃)

### Reaction Step 6

Chemical Formula F (0.25 g, 1.0492 mmol) was dissolved in 20 mL of methanol, benzaldehyde (1.259 mmol) was then added thereto. Potassium hydroxide (KOH, 0.1761 g, 3.1471 mmol) dissolved in 10 mL of methanol was added to the solution, and the resulting mixture was stirred for 48 hours. The reaction product was diluted with 150 mL of ethyl acetate, and neutralized with 100 mL of an aqueous NH₄C₁ solution. The organic solvent layer was washed with water and an aqueous saturated NaCl solution, and moisture was removed using MgSO₄. The solvent was removed by distillation under reduced pressure, and the residue was separated using column chromatography (n-hexane or n-hexane:acetone=300:1) to obtain Chemical Formulas 15 to 18.

### Example 3: Confirmation of DMC Derivatives

Compounds 1 to 18 according to the present invention prepared through the above process are shown in Table 1 below, and their chemical structures were confirmed through spectroscopy analysis.

TABLE 1

| Name | IUPAC Name | Structral Formula |
|---|---|---|
| Compound 1 | 1-(2′-hydroxy-4′(methoxymethoxy)-6′- methoxy-3′,5′-dimethylphenyl)-3-(3- fluorophenyl)-2-propen-1-one | 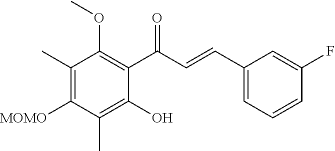 |
| Compound 2 | 1-(2′-hydroxy-4′-(methoxymethoxy)-6′- methoxy-3′,5′-dimethylphenyl)-3-(3,4- difluorophenyl)-2-propen-1-one | 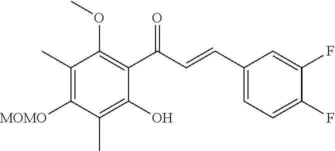 |
| Compound 3 | 1-(2-hydroxy-4′-(methoxymethoxy)-6′- methoxy-3′,5′-dimethylphenyl)-3-(3- trifluoromethylphenyl)-2-propen-1-one | 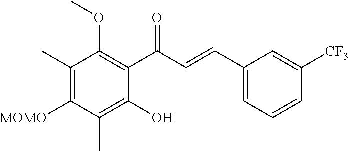 |
| Compound 4 | 1-(2′-hydroxy-4′-(methoxymethoxy)-6′- methoxy-3′,5′-dimethylphenyl)-3-(2,5- difluorophenyl)-2-propen-1-one | 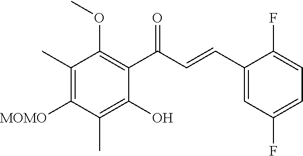 |
| Compound 5 | 1-(2′-hydroxy-4′-(methoxymethoxy)-6′ methoxy-3′,5′-dimethylphenyl)-3-(3,5- difluorophenyl)-2-propen-1-one | 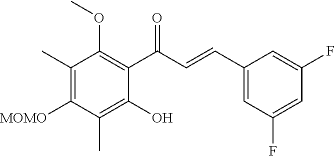 |
| Compound 6 | 1-(2′-hydroxy-4′-(methoxymethoxy)-6′- methoxy-3′,5′-dimethylphenyl)-3-(3,5- dimethylphenyl)-2-propen-1-one | 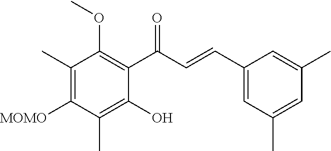 |
| Compound 7 | 1-(2′-hydroxy-4′-(methoxymethoxy)-6′- methoxy-3′,5′-dimethylphenyl)-3-(3- bromophenyl)-2-propen-1-one | 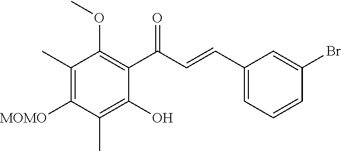 |
| Compound 8 | 1-(2′,4′-dihydroxy-6′-methoxy-3′,5′- dimethylpheny!)-3-(3-fluorophenyl)-2- propen-1-one | 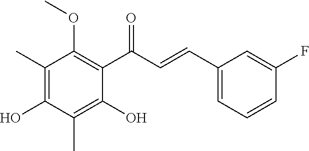 [SMILES: COc1c(C)c(O)c(C)c(O)c1C(=O)/C=C/c1cccc(F)c1] |
| Compound 9 | 1-(2′,4-dihydroxy-6′-methoxy-3′,5′- dimethylphenyl)-3-(3,4-difluorophenyl)-2- propen-1-one | 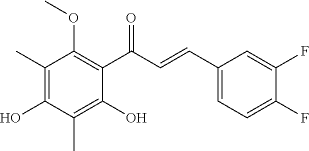 [SMILES: COc1c(C)c(O)c(C)c(O)c1C(=O)/C=C/c1ccc(F)c(F)c1] |
| Compound 10 | 1-(2′,4′-dihydroxy-6′-methoxy-3′,5′- dimethylphenyl)-3-(3- trifluoromethylphenyl)-2-propen-1-one | 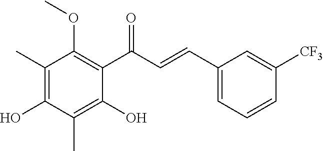 [SMILES: COc1c(C)c(O)c(C)c(O)c1C(=O)/C=C/c1cccc(C(F)(F)F)c1] |
| Compound 11 | 1-(2′,4′-dihydroxy-6′-methoxy-3′,5′- dimethylphenyl)-3-(2,5-difluorophenyl)-2- propen-1-one | 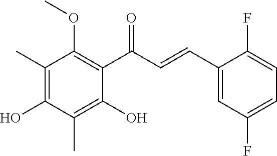 [SMILES: COc1c(C)c(O)c(C)c(O)c1C(=O)/C=C/c1cc(F)ccc1F] |
| Compound 12 | 1-(2′,4′-dihydroxy-6′-methoxy-3′,5′- dimethylphenyl)-3-(3,5-difluorophenyl)-2- propen-1-one | 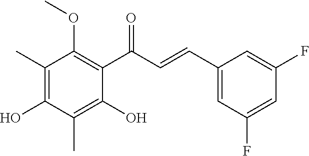 [SMILES: COc1c(C)c(O)c(C)c(O)c1C(=O)/C=C/c1cc(F)cc(F)c1] |
| Compound 13 | 1-(2′,4′-dihydroxy-6′-methoxy-3′,5′- dimethylphenyl)-3-(3,5-dimethylphenyl)-2- propen-1-one | 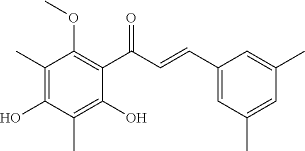 |
| Compound 14 | 1-(2′,4′-dihydroxy-6′ -methoxy-3′,5′- dimethylphenyl)-3-(3-bromophenyl)-2- propen-1-one | 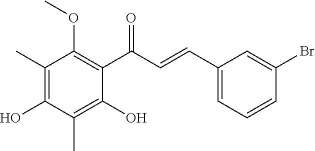 [SMILES: COc1c(C)c(O)c(C)c(O)c1C(=O)/C=C/c1cccc(Br)c1] |
| Compound 15 | 1-(2′,4′,6′-trimethoxy-3′,5′- dimethylphenyl)-3-(3-fluorophenyl)-2- propen-1-one | 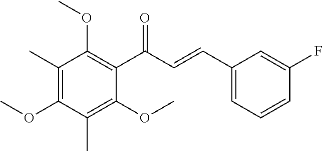 [SMILES: COc1c(C)c(OC)c(C(=O)/C=C/c2cccc(F)c2)c(OC)c1C] |
| Compound 16 | 1-(2′,4′,6′trimethoxy-3′,5′ dimethylphenyl)-3-(3,4-difluorophenyl)~2- propen-1-one | 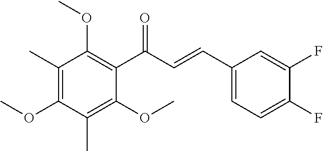 [SMILES: COc1c(C)c(OC)c(C(=O)/C=C/c2ccc(F)c(F)c2)c(OC)c1C] |
| Compound 17 | 1-(2′,4′,6′-strimethoxy-3′,5′- dimethylphenyl)-3-(3- (trifluoromethyl)phenyl)-2-propen-1-one | 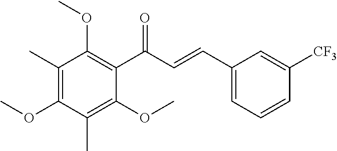 [SMILES: COc1c(C)c(OC)c(C(=O)/C=C/c2cccc(C(F)(F)F)c2)c(OC)c1C] |
| Compound 18 | 1-(2′,4′,6′-trimethoxy-3′,5′- dimethylphenyl)-3-(2-chloro-5- (trifluoromethyl)phenyl)-2-propen-1-one | 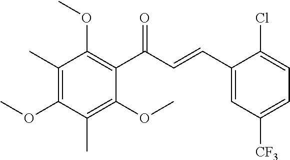 [SMILES: COc1c(C)c(OC)c(C(=O)/C=C/c2cc(C(F)(F)F)ccc2Cl)c(OC)c1C] |

### 3-1. Analysis Data of 1-(2′-hydroxy-4′-(methoxymethoxy)-6′-methoxy-3′,5′-dimethylphenyl)-3-(3-fluorophenyl)-2-propen-1-one (Compound 1)

¹H NMR (DMSO-d₆, 600 MHz) δ11.83 (s, 1H, OH), 7.70 (d, J=15.82 Hz, 1H, —C═C—H), 7.65 (d, J=15.82 Hz, 1H, —C═C—H), 7.62 (dd, J=10.15 Hz, J=2.33 Hz 1H, Ar—H), 7.59 (d, J=7.78 Hz, 1H, Ar—H), 7.49 (ddd, J=8.15 Hz, J=7.78 Hz, J=5.98 Hz, 1H, Ar—H), 7.28 (ddd, J=10.89 Hz, J=8.15 Hz, J=2.33 Hz, 1H, Ar—H), 5.02 (s, 2H, —CH₂), 3.61 (s, 3H, —OCH₃), 3.52 (s, 3H, —OCH₃), 2.11 (s, 3H, —CH₃), 2.08 (s, 3H, —CH₃); ¹³C NMR (DMSO-d₆, 150 MHz) δ193.7 (1C), 162.4 (d, J=244.14 Hz, 1C), 160.1 (1C), 157.8 (1C), 157.1 (1C), 141.7 (1C), 137.5 (d, J=8 Hz, 1C), 131.0 (d, J=8.4 Hz, 1C), 128.4 (1C), 124.7 (d, J=2.54 Hz, 1C), 117.2 (d, J=21.35 Hz, 1C), 115.7 (1C), 115.0 (1C), 114.8 (d, J=21.93 Hz, 1C), 113.8 (1C), 99.0 (1C), 61.9 (1C), 57.1 (1C), 9.5 (1C), 9.4 (1C)

### 3-2. Analysis Data of 1-(2′-hydroxy-4′-(methoxymethoxy)-6′-methoxy-3′,5′-dimethylphenyl)-3-(3,4-difluorophenyl)-2-propen-1-one (Compound 2)

¹H NMR (CDCl₃, 600 MHz) δ12.95 (s, 1H, OH), 7.86 (d, J=15.64 Hz, 1H, —C═C—H), 7.74 (d, J=15.64 Hz, 1H, —C═C—H), 7.46 (ddd, J=11.02 Hz, J=7.63 Hz, J=2.00 Hz, 1H, Ar—H), 7.37-7.35 (m, 1H, Ar—H), 7.21 (ddd, J=9.92 Hz, J=8.27 Hz, J=8.21 Hz, 1H, Ar—H), 5.02 (s, 2H, —CH₂), 3.65 (s, 3H, —OCH₃), 3.63 (s, 3H, —OCH₃), 2.19 (s, 3H, —CH₃), 2.17 (s, 3H, —CH₃).

### 3-3. Analysis Data of 1-(2′-hydroxy-4′-(methoxymethoxy)-6′-methoxy-3′,5′-dimethylphenyl)-3-(3-(trifluoromethyl)phenyl)-2-propen-1-one (Compound 3)

¹H NMR (CDCl₃, 600 MHz) δ12.96 (s, 1H, OH), 7.99 (d, J=15.72 Hz, 1H, —C═C—H), 7.86 (s, 1H, Ar—H), 7.84 (d, J=15.72 Hz, 1H, —C═C—H), 7.81 (d, J=7.72 Hz, 1H, Ar—H), 7.65 (d, J=7.80 Hz, 1H, Ar—H), 7.55 (t, J=7.75 Hz, 1H, Ar—H), 5.03 (s, 2H, —CH₂), 3.66 (s, 3H, —OCH₃), 3.63 (s, 3H, —OCH₃), 2.20 (s, 3H, —CH₃), 2.18 (s, 3H, —CH₃).

### 3-4. Analysis Data of 1-(2′-hydroxy-4′-(methoxymethoxy)-6′-methoxy-3′,5′-dimethylphenyl)-3-(2,5-difluorophenyl)-2-propen-1-one (Compound 4)

¹H NMR (CDCl₃, 600 MHz) δ12.99 (s, 1H, OH), 7.99 (d, J=15.64 Hz, 1H, —C═C—H), 7.90 (d, J=15.64 Hz, 1H, —C═C—H), 7.36-7.32 (m, 1H, Ar—H), 7.12-7.04 (m, 2H, Ar—H), 5.02 (s, 2H, —CH₂), 3.66 (s, 3H, —OCH₃), 3.62 (s, 3H, —OCH₃), 2.19 (s, 3H, —CH₃), 2.17 (s, 3H, —CH₃).

### 3-5. Analysis Data of 1-(2′-hydroxy-4′-(methoxymethoxy)-6′-methoxy-3′,5′-dimethylphenyl)-3-(3,5-difluorophenyl)-2-propen-1-one (Compound 5)

¹H NMR (CDCl₃, 600 MHz) δ12.91 (s, 1H, OH), 7.91 (d, J=15.60 Hz, 1H, —C═C—H), 7.70 (d, J=15.60 Hz, 1H, —C═C—H), 7.13 (dt, J=6.07 Hz, J=1.84 Hz, 2H, Ar—H), 6.85 (tt, J=8.67 Hz, J=2.33 Hz, 1H, Ar—H), 5.02 (s, 2H, —CH₂), 4.12 (q, J=6.99 Hz, 2H, —CH₂), 3.65 (s, 3H, —OCH₃), 3.62 (s, 3H, —OCH₃), 2.19 (s, 3H, —CH₃), 2.17 (s, 3H, —CH₃).

### 3-6. Analysis Data of 1-(2′-hydroxy-4′-(methoxymethoxy)-6′-methoxy-3′,5′-dimethylphenyl)-3-(3,5-dimethylphenyl)-2-propen-1-one (Compound 6)

¹H NMR (DMSO-d₆, 600 MHz) δ11.94 (s, 1H, OH), 7.65 (d, J=15.83 Hz, 1H, —C═C—H), 7.61 (d, J=15.83 Hz, 1H, —C═C—H), 7.34 (s, 2H, Ar—H), 7.10 (s, 1H, Ar—H), 5.02 (s, 2H, —CH₂), 3.61 (s, 3H, —OCH₃), 3.52 (s, 3H, —OCH₃), 2.31 (s, 6H, —CH₃), 2.12 (s, 3H, —CH₃), 2.07 (s, 3H, —CH₃); ¹³C NMR (DMSO-d₆, 150 MHz) δ193.8 (1C), 160.0 (1C), 157.9 (1C), 157.1 (1C), 140.0 (1C), 138.2 (2C), 134.4 (1C), 132.3 (1C), 126.5 (1C), 126.3 (2C), 115.7 (1C), 115.0 (1C), 113.8 (1C), 99.0 (1C), 62.0 (1C), 57.1 (1C), 20.7 (2C), 9.5 (1C), 9.5 (1C).

### 3-7. Analysis Data of 1-(2′-hydroxy-4′-(methoxymethoxy)-6′-methoxy-3′,5′-dimethylphenyl)-3-(3-bromophenyl)-2-propen-1-one (Compound 7)

¹H NMR (CDCl₃, 600 MHz) δ12.97 (s, 1H, OH), 7.92 (d, J=15.64 Hz, 1H, —C═C—H), 7.78 (s, 1H, Ar—H), 7.75 (d, J=15.64 Hz, 1H, —C═C—H), 7.55 (d, J=7.85 Hz, 1H, Ar—H), 7.52 (d, J=7.93 Hz, 1H, Ar—H), 7.29 (t, J=7.85 Hz, 1H, Ar—H), 5.02 (s, 2H, —CH₂), 3.66 (s, 3H, —OCH₃), 3.63 (s, 3H, —OCH₃), 2.20 (s, 3H, —CH₃), 2.17 (s, 3H, —CH₃); ¹³C NMR (CDCl₃, 150 MHz) δ193.9 (1C), 162.0 (1C), 161.7 (1C), 158.8 (1C), 141.6 (1C), 137.6 (1C), 133.6 (1C), 131.6 (1C), 130.7 (1C), 128.1 (1C), 127.4 (1C), 123.3 (1C), 116.2 (1C), 116.1 (1C), 112.1 (1C), 99.5 (1C), 62.6 (1C), 58.0 (1C), 9.8 (1C), 9.7 (1C).

### 3-8. Analysis Data of 1-(2′,4′-dihydroxy-6′-methoxy-3′,5′-dimethylphenyl)-3-(3-fluorophenyl)-2-propen-1-one (Compound 8)

¹H NMR (CDCl₃, 600 MHz) δ13.51 (s, 1H, OH), 7.96 (d, J=15.66 Hz, 1H, —C═C—H), 7.77 (d, J=15.66 Hz, 1H, —C═C—H), 7.41-7.36 (m, 2H, Ar—H), 7.34 (d, J=9.67 Hz, 1H, Ar—H), 7.11-7.07 (m, 1H, Ar—H), 5.37 (s, 1H, OH), 3.66 (s, 3H, —OCH₃), 2.16 (s, 3H, —CH₃), 2.13 (s, 3H, —CH₃); ¹³C NMR (CDCl₃, 150 MHz) δ193.3 (1C), 163.3 (d, J=243.91, 1C), 162.3 (1C), 159.6 (1C), 159.1 (1C), 141.4 (d, J=1.65, 1C), 137.9 (d, J=7.75, 1C), 130.6 (d, J=8.4, 1C), 128.3 (1C), 124.7 (d, J=2.28, 1C), 117.2 (d, J=20.68, 1C), 114.6 (d, J=21.84, 1C), 109.2 (1C), 109.1 (1C), 106.8 (1C), 62.6 (1C), 8.4 (1C), 7.7 (1C).

### 3-9. Analysis Data of 1-(2′,4′-dihydroxy-6′-methoxy-3′,5′-dimethylphenyl)-3-(3,4-difluorophenyl)-2-propen-1-one (Compound 9)

¹H NMR (CDCl₃, 600 MHz) δ13.47 (s, 1H, OH), 7.88 (d, J=15.69 Hz, 1H, —C═C—H), 7.71 (d, J=15.69 Hz, 1H, —C═C—H), 7.45 (ddd, J=10.93 Hz, J=7.57 Hz, J=1.95 Hz 1H, Ar—H), 7.34-7.36 (m, 1H, Ar—H), 7.20 (ddd, J=10.00 Hz, J=8.33 Hz, J=8.20 Hz, Ar—H), 5.42 (s, 1H, OH), 3.65 (s, 3H, —OCH₃), 2.15 (s, 3H, —CH₃), 2.13 (s, 3H, —CH₃).

### 3-10. Analysis Data of 1-(2′,4′-dihydroxy-6′-methoxy-3′,5′-dimethylphenyl)-3-(3-(trifluoromethyl)phenyl)-2-propen-1-one (Compound 10)

¹H NMR (CDCl₃, 600 MHz) δ13.48 (s, 1H, OH), 8.01 (d, J=15.68 Hz, 1H, —C═C—H), 7.85 (s, 1H, Ar—H), 7.82 (d, J=15.68 Hz, 1H, —C═C—H), 7.81 (d, J=8.51 Hz, 1H, Ar—H), 7.64 (d, J=7.79 Hz, 1H, Ar—H), 7.54 (t, J=7.77 Hz, 1H, Ar—H), 5.36 (s, 1H, OH), 3.66 (s, 3H, —OCH₃), 2.16 (s, 3H, —CH₃), 2.14 (s, 3H, —CH₃); ¹³C NMR (CDCl₃, 150 MHz) δ193.1 (1C), 162.3 (1C), 159.7 (1C), 159.1 (1C), 140.9 (1C), 136.4 (1C), 131.5 (1C), 129.7 (1C), 128.8 (1C), 126.7 (q, J=3.65 Hz, 1C), 125.0 (q, J=3.67 Hz, 1C), 109.2 (1C), 109.2 (1C), 106.8 (1C), 62.6 (1C), 8.4 (1C), 7.7 (1C).

### 3-11. Analysis Data of 1-(2′,4′-dihydroxy-6′-methoxy-3′,5′-dimethylphenyl)-3-(2,5-difluorophenyl)-2-propen-1-one (Compound 11)

¹H NMR (CDCl₃, 600 MHz) δ13.51 (s, 1H, OH), 8.01 (d, J=15.84 Hz, 1H, —C═C—H), 7.88 (d, J=15.84 Hz, 1H, —C═C—H), 7.36-7.32 (m, 1H, Ar—H), 7.11-7.03 (m, 2H, Ar—H), 5.34 (s, 1H, OH), 3.66 (s, 3H, —OCH₃), 3.66 (s, 3H, —OCH₃), 2.15 (s, 3H, —CH₃), 2.13 (s, 3H, —CH₃).

### 3-12. Analysis Data of 1-(2′,4′-dihydroxy-6′-methoxy-3′,5′-dimethylphenyl)-3-(3,5-difluorophenyl)-2-propen-1-one (Compound 12)

¹H NMR (CDCl₃, 600 MHz) δ13.42 (s, 1H, OH), 7.93 (d, J=15.69 Hz, 1H, —C═C—H), 7.67 (d, J=15.69 Hz, 1H, —C═C—H), 7.13 (dt, J=10.93 Hz, J=1.95 Hz, 2H, Ar—H), 7.20 (tt, J=10.00 Hz, J=8.20 Hz, 1H, Ar—H), 5.38 (s, 1H, OH), 3.65 (s, 3H, —OCH₃), 2.16 (s, 3H, —CH₃), 2.13 (s, 3H, —CH₃).

### 3-13. Analysis Data of 1-(2′,4′-dihydroxy-6′-methoxy-3′,5′-dimethylphenyl)-3-(3,5-dimethylphenyl)-2-propen-1-one (Compound 13)

¹H NMR (DMSO-d₆, 600 MHz) δ13.59 (s, 1H, OH), 9.64 (s, 1H, OH), 7.88 (d, J=15.67 Hz, 1H, —C═C—H), 7.69 k (d, J=15.67 Hz, 1H, —C═C—H), 7.32 (s, 2H, Ar—H), 7.09 (s, 1H, Ar—H), 3.60 (s, 3H, —OCH₃), 2.32 (s, 6H, —CH₃), 2.07 (s, 3H, —CH₃), 2.02 (s, 3H, —CH₃); ¹³C NMR (DMSO-d₆, 150 MHz) δ192.5 (1C), 161.2 (1C), 161.1 (1C), 158.2 (1C), 142.8 (1C), 138.2 (2C), 134.8 (1C), 132.1 (1C), 126.2 (1C), 126.1 (2C), 110.1 (1C), 107.9 (1C), 107.1 (1C), 61.9 (1C), 20.8 (2C), 8.93 (1C), 8.32 (1C).

### 3-14. Analysis Data of 1-(2′,4′-dihydroxy-6′-methoxy-3′,5′-dimethylphenyl)-3-(3-bromophenyl)-2-propen-1-one (Compound 14)

¹H NMR (CDCl₃, 600 MHz) δ 13.49 (s, 1H, OH), 7.94 (d, J=15.66 Hz, 1H, —C═C—H), 7.76 (s, 1H, Ar—H), 7.72 (d, J=15.66 Hz, 1H, —C═C—H), 7.54 (d, J=7.74 Hz, 1H, Ar—H), 7.51 (d, J=7.92 Hz, 1H, Ar—H), 7.28 (t, J=7.84 Hz, 1H, Ar—H), 5.49 (s, 1H, OH), 3.65 (s, 3H, —OCH₃), 2.15 (s, 3H, —CH₃), 2.13 (s, 3H, —CH₃); ¹³C NMR (CDCl₃, 150 MHz) δ 193.2 (1C), 162.3 (1C), 159.7 (1C), 159.0 (1C), 141.1 (1C), 137.7 (1C), 133.1 (1C), 131.0 (1C), 130.6 (1C), 128.3 (1C), 127.2 (1C), 123.3 (1C), 109.2 (1C), 109.2 (1C), 106.8 (1C), 62.6 (1C), 8.4 (1C), 7.7 (1C).

### 3-15. Analysis Data of 1-(2′,4′,6′-trimethoxy-3′,5′-dimethylphenyl)-3-(3-fluorophenyl)-2-propen-1-one (Compound 15)

¹H NMR (CDCl₃, 600 MHz) δ 7.34-7.28 (m, 1H, Ar—H), 7.32 (d, J=15.84 Hz, 1H, —C═C—H), 7.29 (d, J=8.63 Hz, 1H, Ar—H), 7.23 (dd, J=9.57 Hz, J=1.95 Hz, 1H, Ar—H), 7.07-7.02 (m, 1H, Ar—H), 7.02 (d, J=15.84 Hz, 1H, —C═C—H), 3.74 (s, 3H, —OCH₃), 3.68 (s, 6H, —OCH₃), 2.20 (s, 6H, —CH₃); ¹³C NMR (CDCl₃, 150 MHz) δ 194.6 (1C), 163.0 (d, J=246.83 Hz, 1C), 159.4 (1C), 154.6 (1C), 143.7 (1C), 136.9 (d, J=7.65 Hz, 1C), 130.4 (d, J=8.18 Hz, 1C), 129.6 (2C), 124.7 (1C), 124.5 (d, J=2.84 Hz, 1C), 120.9 (2C), 117.3 (d, J=21.42 Hz, 1C), 114.7 (d, J=21.90 Hz, 1C), 62.2 (2C), 60.0 (1C), 9.3 (2C).

### 3-16. Analysis Data of 1-(2′,4′,6′-trimethoxy-3′,5′-dimethylphenyl)-3-(3,4-difluorophenyl)-2-propen-1-one (Compound 16)

¹H NMR (CDCl₃, 600 MHz) δ 7.36 (t, J=8.60 Hz, 1H, Ar—H), 7.26 (d, J=16.04 Hz, 1H, —C═C—H), 7.24-7.28 (m, 1H, Ar—H), 7.15 (q, J=8.81 Hz, 1H, Ar—H), 6.93 (d, J=16.04 Hz, 1H, —C═C—H), 3.76 (s, 3H, —OCH₃), 3.69 (s, 6H, —OCH₃), 2.21 (s, 6H, —CH₃); ¹³C NMR (CDCl₃, 150 MHz) δ 194.6 (1C), 159.7 (1C), 154.7 (1C), 151.7 (dd, J=253.73 Hz, J=12.96 Hz, 1C), 150.8 (dd, J=249.28 Hz, J=13.19 Hz, 1C), 142.8 (1C), 132.1 (dd, J=5.62 Hz, J=4.49 Hz, 1C), 129.4 (d, J=2.50 Hz, 1C), 125.4 (dd, J=6.60 Hz, J=3.37 Hz, 1C), 124.8 (1C), 121.1 (2C), 117.9 (d, J=17.81 Hz, 1C), 116.9 (d, J=17.40 Hz, 1C), 62.4 (2C), 60.2 (1C), 9.4 (2C).

### 3-17. Analysis Data of 1-(2′,4′,6′-trimethoxy-3′,5′-dimethylphenyl)-3-(3-(trifluoromethyl)phenyl)-2-propen-1-one (Compound 17)

¹H NMR (CDCl₃, 600 MHz) δ 7.75 (s, 1H, Ar—H), 7.72 (d, J=7.77 Hz, 1H, Ar—H), 7.61 (d, J=7.47 Hz, 1H, Ar—H), 7.50 (t, J=7.78 Hz, 1H, Ar—H), 7.36 (d, J=16.12 Hz, 1H, —C═C—H), 7.07 (d, J=16.12 Hz, 1H, —C═C—H), 3.76 (s, 3H, —OCH₃), 3.69 (s, 6H, —OCH₃), 2.21 (s, 6H, —CH₃); ¹³C NMR (CDCl₃, 150 MHz) δ 194.7 (1C), 159.7 (1C), 154.7 (1C), 143.4 (1C), 135.7 (1C), 131.6 (q, J=31.51 Hz, 1C), 131.5 (d, J=1.35 Hz, 1C), 130.1 (1C), 129.6 (2C), 126.9 (q, J=3.82 Hz, 1C), 125.3 (q, J=3.77 Hz, 1C), 124.8 (1C), 123.9 (q, J=272.44 Hz, 1C), 121.1 (2C), 62.4 (2C), 60.2 (1C), 9.4 (2C).

### 3-18. Analysis Data of 1-(2′,4′,6′-trimethoxy-3′,5′-dimethylphenyl)-3-(2-chloro-5-(trifluoromethyl)phenyl)-2-propen-1-one (Compound 18)

¹H NMR (CDCl₃, 600 MHz) δ 7.90 (br-d, J=1.85 Hz, 1H, Ar—H), 7.82 (d, J=16.09 Hz, 1H, —C═C—H), 7.54 (dd, J=8.10 Hz J=1.85 Hz, 1H, Ar—H), 7.52 (d, J=8.10 Hz, 1H, Ar—H), 7.07 (d, J=16.09 Hz, 1H, —C═C—H), 3.75 (s, 3H, —OCH₃), 3.70 (s, 6H, —OCH₃), 2.21 (s, 6H, —CH₃); ¹³C NMR (CDCl₃, 150 MHz) δ 193.9 (1C), 159.8 (1C), 154.9 (2C), 138.7 (d, J=1.47 Hz, 1C), 138.6 (1C), 134.0 (1C), 131.9 (1C), 130.9 (1C), 129.8 (q, J=33.23 Hz, 1C), 127.4 (q, J=3.53 Hz, 1C), 124.8 (q, J=3.77 Hz, 1C), 124.6 (1C), 123.5 (d, J=272.52 Hz, 1C), 121.1 (2C), 62.4 (2C), 60.2 (1C), 9.3 (2C).

### Example 4: Confirmation of Effect of DMC Derivatives on Fatty Acid Oxidation

When fatty acid metabolism is abnormal, neutral fats increase and high-density cholesterol decreases, thereby increasing the risk of metabolic diseases and cardiovascular diseases. Therefore, it was confirmed whether the DMC derivatives of the present invention synthesized according to Example 1 or 2 have an effect on fatty acid oxidation.

To measure the fatty acid oxidation rate, C2C12 myoblasts were primarily cultured in DMEM supplemented with 10% fetal bovine serum, and then cultured for 5 days in DMEM supplemented with 2% horse serum in order to differentiate into myotubes. Then, the differentiated myotubes were treated with 10 μM of the DMC derivative, and cultured for 24 hours, and the cells were then harvested. Next, the harvested cells were lysed using a mitochondrial isolation buffer (250 mmol/L sucrose, 10 mmol/L Tris-HCl, and 1 mmol/L EDTA), treated with 0.2 mmol/L [1-¹⁴C] palmitate, and then allowed to react for 3 hours. Next, the amount of ¹⁴CO₂ thus generated was measured, and the measured amount of ¹⁴CO₂ was corrected for the total amount of proteins to compare the fatty acid oxidation rate. In this case, AICAR was used as a positive control, and the compounds shown in Table 2 below were used as control compounds.

TABLE 2

| Name | Structural Formula |
|---|---|
| CON1 | 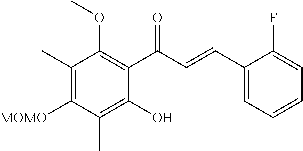 |
| CON2 | 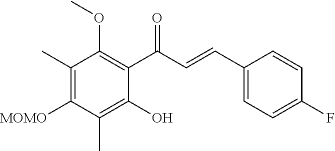 |
| CON3 | 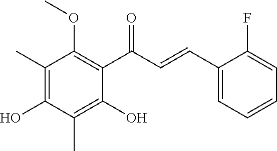 [SMILES: COc1c(C)c(O)c(C)c(O)c1C(=O)/C=C/c1ccccc1F] |
| CON4 | 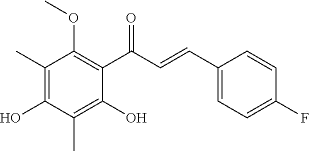 [SMILES: COc1c(C)c(O)c(C)c(O)c1C(=O)/C=C/c1ccc(F)cc1] |
| CON5 | 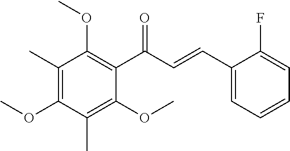 [SMILES: COc1c(C)c(OC)c(C(=O)/C=C/c2ccccc2F)c(OC)c1C] |
| CON6 | 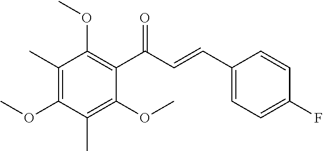 [SMILES: COc1c(C)c(OC)c(C(=O)/C=C/c2ccc(F)cc2)c(OC)c1C] |

As a result, it was confirmed that the DMC derivatives (Compounds 1 to 18) according to the present invention exhibited fatty acid oxidation rates similar to or better than the positive control AICAR, and exhibited higher levels of fatty acid oxidation rates compared to the control compounds.

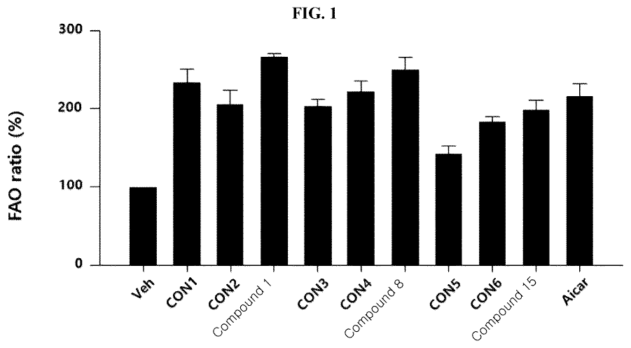

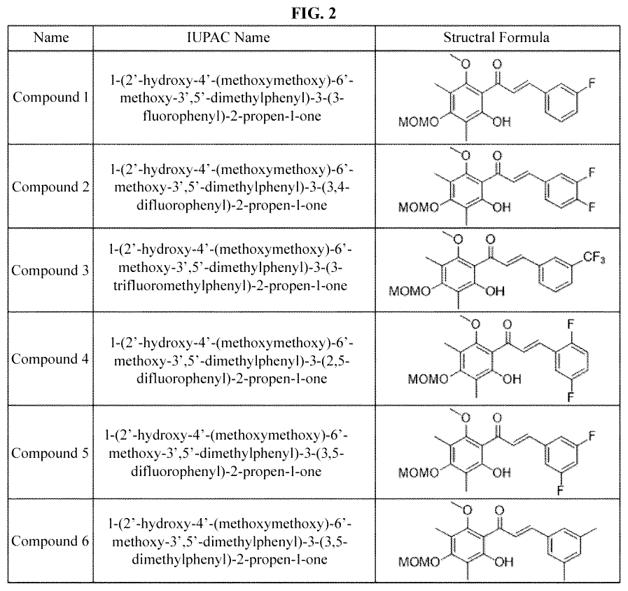

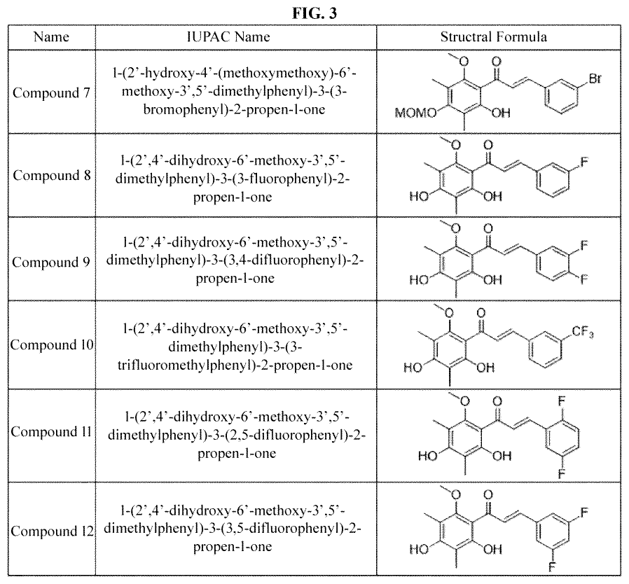

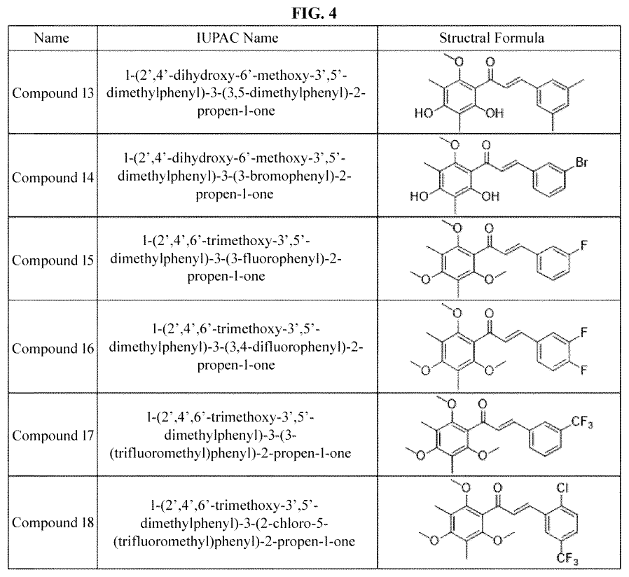

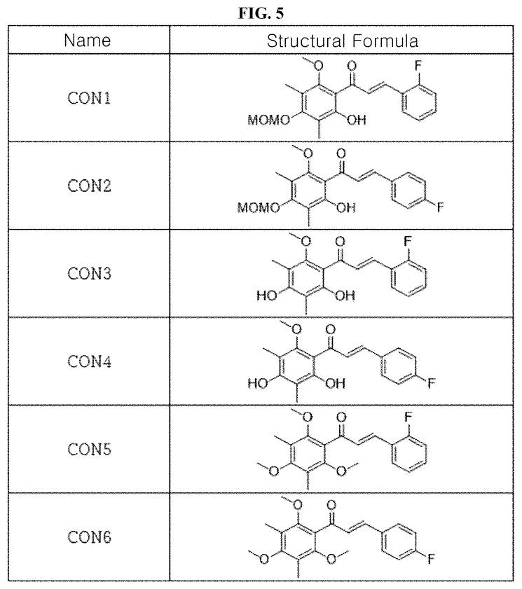

More specifically, when the right ring of the two ring structure of [Chemical Formula I] according to this specification is referred to as “A-ring” and the left ring is referred to as “B-ring,” the derivative (Compound 8) in which the fluorine substituent is bonded to the meta-position on the right ring (A-ring) exhibited a higher fatty acid oxidation effect than the derivatives (CON3, CON4) in which the fluorine substituent is bonded to the ortho- or para-position, as shown in FIG. 1. The structure of the left ring (B-ring) is the same between the control derivatives and the derivatives according to the present invention, which shows that the substituent at the meta-position on the right ring (A-ring) plays a very important role in enhancing the fatty acid oxidation effect.

This phenomenon was observed equally even when the structure of the left ring (B-ring) was changed. That is, even when a methoxymethoxy group was introduced to the left ring (B-ring), the derivative (Compound 1) in which the fluorine substituent was bonded to the meta-position on the right ring (A-ring) exhibited a higher fatty acid oxidation effect than the derivatives (CON1, CON2) in which the fluorine substituent was bonded to the ortho- or para-position. Also, even when three methoxy groups were introduced to the left ring (B-ring), the derivative (Compound 15) in which the fluorine substituent was bonded to the meta-position on the right ring (A-ring) exhibited a higher fatty acid oxidation effect than the derivatives (CON5, CON6) in which the fluorine substituent was bonded to the ortho- or para-position. The structure of the left ring (B-ring) is the same between the control derivatives and the derivatives according to the present invention, which consistently shows that the substituent at the meta-position plays a very important role in enhancing the fatty acid oxidation effect.

In conclusion, the above experimental results show that the derivatives substituted at the meta-position on the right ring (A-ring) exhibit relatively superior fatty acid oxidation effects compared to derivatives substituted at the ortho- or para-positions, regardless of the structure of the left ring (B-ring).

The above results show that the DMC derivatives according to the present invention have excellent fatty acid oxidation effects and may effectively suppress the progression of metabolic diseases including diabetes. In particular, when the structural characteristics are compared with those of the control compound, it is judged that the excellent fatty acid oxidation effects of the DMC derivatives according to the present invention are due to the meta-substitution of the right ring (A-ring) characteristic of the derivatives.

The description of the present invention described above is for illustrative purposes, and it should be understood that those of ordinary skill in the art to which the present invention pertains can easily modify embodiments into other specific forms without changing the technical idea or essential features of the present invention. Therefore, it should be understood that all the embodiments described above are illustrative in all respects and not restrictive.

### INDUSTRIAL APPLICABILITY

The present invention relates to novel compounds based on the meta-substitution of 2′,4′-dihydroxy-6′-methoxy-3′,5′-dimethylchalcone (DMC). According to the present invention, novel meta-substituted DMC derivatives can be simply and efficiently synthesized using inexpensive phloroglucinol or the like as a starting material. In particular, the DMC derivatives according to the present invention have been confirmed to have a superior fatty acid oxidation effect compared to existing DMC derivatives, and thus are expected to be used in the field of treatment of various diseases such as metabolic diseases and the like.

1. A compound represented by the following Chemical Formula I, an isomer thereof, or a pharmaceutically acceptable salt thereof: 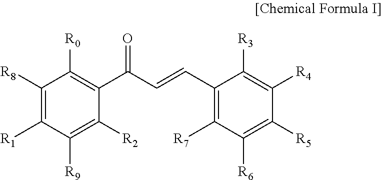
wherein:
R₀, R₁, and R₂ are each independently a hydroxyl group (OH), a methoxymethoxy group (OCH₂OCH₃; OMOM), or a C₁-C₁₀ alkoxy group;
R₈ and R₉ are each independently a C₁-C₁₀ alkyl group;
R₃ to R₇ are each independently any one selected from the group consisting of a halogen, hydrogen (H), deuterium (D), a thiol group (SH), a cyano group (CN), a nitro group (NO₂), a substituted or unsubstituted amino group (NH₂), a substituted or unsubstituted C₁-C₁₀ alkylthio group, a substituted or unsubstituted C₁-C₁₀ alkylsulfonyl group, a substituted or unsubstituted C₁-C₁₀ alkylsulfoxy group, a substituted or unsubstituted C₁-C₁₀ alkyl group, a substituted or unsubstituted C₂-C₁₀ alkenyl group, a substituted or unsubstituted C₂-C₁₀ alkynyl group, and a substituted or unsubstituted C₆-C₂₀ aryl group; and
the term “substituted or unsubstituted” refers to being unsubstituted or substituted with one or more substituents selected from the group consisting of a halogen group, a nitrile group, a nitro group, a hydroxyl group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, an alkylsulfonyl group, an arylsulfonyl group, a silyl group, a boron group, an alkyl group, a cycloalkyl group, an alkenyl group, an alkynyl group, an aryl group, an aralkyl group, an aralkenyl group, an alkylaryl group, an alkylamine group, an aralkylamine group, a heteroarylamine group, an arylamine group, an arylphosphine group, and a heterocyclic group;
provided that at least any one of R₄ and R₆ is not hydrogen;
at least any one of R₀, R₁, and R₂ is not OH; and
when R₀ and R₁ are both OMe or both OMOM, R₂ is not OH or OMOM.

2. The compound, the isomer thereof, or the pharmaceutically acceptable salt thereof of claim 1, wherein R₈ and R₉ are each a methyl group.

3. The compound, the isomer thereof, or the pharmaceutically acceptable salt thereof of claim 1, wherein R₈ and R₉ are each a methyl group, and R₀ is OMe.

4. The compound, the isomer thereof, or the pharmaceutically acceptable salt thereof of claim 1, wherein the compound is represented by the following Chemical Formula 1-1: 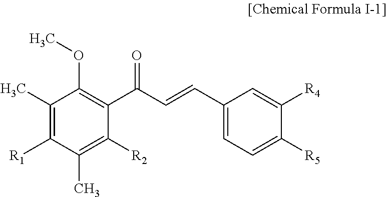
wherein:
R₄ is not hydrogen; and
R₁ and R₂ are each independently OH, OMOM, or a C₁-C₁₀ alkoxy group;
provided that when R₁ is OMe, R₂ is not OH or OMOM.

5. The compound, the isomer thereof, or the pharmaceutically acceptable salt thereof of claim 1, wherein the compound is represented by the following Chemical Formula 1-2: 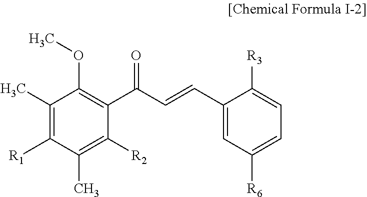
wherein:
R₆ is not hydrogen; and
R₁ and R₂ are each independently OH, OMOM, or a C₁-C₁₀ alkoxy group;
provided that when R₁ is OMe, R₂ is not OH or OMOM.

6. The compound, the isomer thereof, or the pharmaceutically acceptable salt thereof of claim 1, wherein the compound is represented by the following Chemical Formula 1-3: 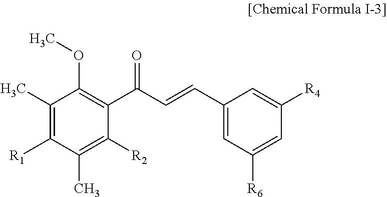
wherein:
R₆ is not hydrogen; and
R₁ and R₂ are each independently OH, OMOM, or a C₁-C₁₀ alkoxy group;
provided that when R₁ is OMe, R₂ is not OH or OMOM.

7. The compound, the isomer thereof, or the pharmaceutically acceptable salt thereof of claim 1, wherein the compound is selected from the group consisting of the following:
(1) 1-(2′-hydroxy-4′-(methoxymethoxy)-6′-methoxy-3′,5′-dimethylphenyl)-3-(3-fluorophenyl)-2-propen-1-one;
(2) 1-(2′-hydroxy-4′-(methoxymethoxy)-6′-methoxy-3′,5′-dimethylphenyl)-3-(3,4-difluorophenyl)-2-propen-1-one;
(3) 1-(2′-hydroxy-4′-(methoxymethoxy)-6′-methoxy-3′,5′-dimethylphenyl)-3-(3-trifluoromethylphenyl)-2-propen-1-one;
(4) 1-(2′-hydroxy-4′-(methoxymethoxy)-6′-methoxy-3′,5′-dimethylphenyl)-3-(2,5-difluorophenyl)-2-propen-1-one;
(5) 1-(2′-hydroxy-4′-(methoxymethoxy)-6′-methoxy-3′,5′-dimethylphenyl)-3-(3,5-difluorophenyl)-2-propen-1-one;
(6) 1-(2′-hydroxy-4′-(methoxymethoxy)-6′-methoxy-3′,5′-dimethylphenyl)-3-(3,5-dimethylphenyl)-2-propen-1-one;
(7) 1-(2′-hydroxy-4′-(methoxymethoxy)-6′-methoxy-3′,5′-dimethylphenyl)-3-(3-bromophenyl)-2-propen-1-one;
(8) 1-(2′,4′-dihydroxy-6′-methoxy-3′,5′-dimethylphenyl)-3-(3-fluorophenyl)-2-propen-1-one;
(9) 1-(2′,4′-dihydroxy-6′-methoxy-3′,5′-dimethylphenyl)-3-(3,4-difluorophenyl)-2-propen-1-one;
(10) 1-(2′,4′-dihydroxy-6′-methoxy-3′,5′-dimethylphenyl)-3-(3-trifluoromethylphenyl)-2-propen-1-one;
(11) 1-(2′,4′-dihydroxy-6′-methoxy-3′,5′-dimethylphenyl)-3-(2,5-difluorophenyl)-2-propen-1-one;
(12) 1-(2′,4′-dihydroxy-6′-methoxy-3′,5′-dimethylphenyl)-3-(3,5-difluorophenyl)-2-propen-1-one;
(13) 1-(2′,4′-dihydroxy-6′-methoxy-3′,5′-dimethylphenyl)-3-(3,5-dimethylphenyl)-2-propen-1-one;
(14) 1-(2′,4′-dihydroxy-6′-methoxy-3′,5′-dimethylphenyl)-3-(3-bromophenyl)-2-propen-1-one;
(15) 1-(2′,4′,6′-trimethoxy-3′,5′-dimethylphenyl)-3-(3-fluorophenyl)-2-propen-1-one;
(16) 1-(2′,4′,6′-trimethoxy-3′,5′-dimethylphenyl)-3-(3,4-difluorophenyl)-2-propen-1-one;
(17) 1-(2′,4′,6′-trimethoxy-3′,5′-dimethylphenyl)-3-(3-(trifluoromethyl)phenyl)-2-propen-1-one; and
(18) 1-(2′,4′,6′-trimethoxy-3′,5′-dimethylphenyl)-3-(2-chloro-5-(trifluoromethyl)phenyl)-2-propen-1-one.

8. A composition for treating a metabolic disease, comprising the compound, the isomer thereof, or the pharmaceutically acceptable salt thereof of claim 1 as an active ingredient.

9. The composition of claim 8, wherein the composition is a pharmaceutical composition, or a food composition.

10. The composition of claim 8, wherein the metabolic disease is any one or more selected from the group consisting of obesity, diabetes, hypertension, hyperlipidemia, arteriosclerosis, acute coronary syndrome, non-alcoholic fatty liver, diabetic retinopathy, diabetic nephropathy, diabetic foot disease, diabetic neuropathy, hyperlipidemia, stroke, angina pectoris, myocardial infarction, and peripheral vascular disease.

11. A method of treating a metabolic disease, comprising:
administering a pharmaceutically effective amount of the compound, the isomer thereof, or the pharmaceutically acceptable salt thereof of claim 1 as an active ingredient to a subject in need thereof.

12. The method of claim 11, wherein the metabolic disease is any one or more selected from the group consisting of obesity, diabetes, hypertension, hyperlipidemia, arteriosclerosis, acute coronary syndrome, non-alcoholic fatty liver, diabetic retinopathy, diabetic nephropathy, diabetic foot disease, diabetic neuropathy, hyperlipidemia, stroke, angina pectoris, myocardial infarction, and peripheral vascular disease.
